SEQUENCE LISTING (14 sequences)

SEQ ID NO: 1 - DNA, 23 bp, Artificial Sequence
  Description of Artificial Sequence oligonucleotide primer
cttctagagactgcagatgtttc

SEQ ID NO: 2 - DNA, 25 bp, Artificial Sequence
  Description of Artificial Sequence oligonucleotide primer
gatgatgctctgagaaaaggtaatc

SEQ ID NO: 3 - DNA, 36 bp, Artificial Sequence
  Description of Artificial Sequence forward primer AB9883
cttaagcttgccaccatggacatgagggtccccgct

SEQ ID NO: 4 - DNA, 35 bp, Artificial Sequence
  Description of Artificial Sequence reverse primer AB7824
ccgaattctttaatctccagttttgtcccttgggc

SEQ ID NO: 5 - DNA, 87 bp, Artificial Sequence
  Description of Artificial Sequence forward primer AB7822
ggggaattcggtggcggtggctcgggcggtggtgggtcgggtggcggcggatctatgcctacttcaagttctacaaagaaaacacag

SEQ ID NO: 6 - protein, 15 aa, Artificial Sequence
  Description of Artificial Sequence linker peptide
GGGGSGGGGSGGGGS

SEQ ID NO: 7 - DNA, 25 bp, Artificial Sequence
  Description of Artificial Sequence reverse primer AB4749
gacctcgagtcagtgttgagatgat

SEQ ID NO: 8 - protein, 133 aa, Artificial Sequence [sequence omitted]

SEQ ID NO: 9 - DNA, 816 bp, Artificial Sequence
  Description of Artificial Sequence 520C9 humanized single-chain antibody used in the IL-2-antibody fusions
ggtaccatggacatgagggtccccgctcagctcctggggctcctgctgctctggttcccaggttctagatgtgagatgcaactggtggagtctgggcctgaggtgaagaagcctggggcctcagtgaaggtctcctgcaaggcttctggttacacctttaccaactatggaatgaactgggtgcgacaggcccctggacaagggcttgagtggatgggatggataaacacctacactggacagtcaacatatgctgatgacttcaaggaaagagtcaccatgaccacagacacatccacgagcacagcctacatggacctgaggagcctgagatctgacgacacggccgtgtattactgtgcgagacgatttgggtttgcttactggggccagggaaccctggtcaccgtctcctcaggtggcggtggctcgggcggtggtgggtcgggtggcggcggatctgacatccagatgacccagtctccatcctccctgtctgcatctgtaggagacagagtcaccatcacttgccgggcaagtcaggacattggtaatagcttaacctggtatcagcagaaaccagggaaaacccctaagctcctgatctacgccacatccagtttagattctggggtcccatcaaggttcagtggaagtggatctgggacagattttactttcaccatcagcagtctgcagcctgaagatattgcaacatattactgtctacaatatgctatttttccgtacacgttcggccaagggacacgactggagattaaaggatccgaatacatgccaatggaatgacccggg

SEQ ID NO: 10 - protein, 267 aa, Artificial Sequence
  Description of Artificial Sequence 520C9 humanized single-chain antibody used in the IL-2-antibody fusions
MDMRVPAQLLGLLLLWFPGSRCEMQLVESGPEVKKPGASVKVSCKASGYTFTNYGMNWVRQAPGQGLEWMGWINTYTGQSTYADDFKERVTMTTDTSTSTAYMDLRSLRSDDTAVYYCARRFGFAYWGQGTLVTVSSGGGGSGGGGSGGGGSDIQMTQSPSSLSASVGDRVTITCRASQDIGNSLTWYQQKPGKTPKLLIYATSSLDSGVPSRFSGSGSGTDFTFTISSLQPEDIATYYCLQYAIFPYTFGQGTRLEIKGSEYMPME

SEQ ID NO: 11 - DNA, 840 bp, Artificial Sequence
  Description of Artificial Sequence H520C9sFv plus linker
aagcttgccaccatggacatgagggtccccgctcagctcctggggctcctgctgctctggttcccaggttctagatgtgagatacaactggtgcagtctgggcctgaggtgaagaagcctggggcctcagtgaagatctcctgcaaggcttctggttacacctttgccaactatggaatgaactggatgaaacaggcccctggaaaagggcttgagtggatgggatggataaacacctacactggacagtcaacatatgctgatgacttcaaggaaagattcaccttcaccctagacacatccacgagcacagcccacctggaaataagcagcctgagatctgaggacacggccacgtatttctgtgcgagacgatttgggtttgcttactggggccagggaaccctggtcaccgtctcctcaggtggcggtggctcgggcggtggtgggtcgggtggcggcggatctgacatccagatgacccagtctccatcctccctgtctgcatctgtaggagacagagtcaccctcacttgccgggcaagtcaggacattggtaatagcttaacctggttacagcagaaaccagggaaaaccattaagcgcctgatctacgccacatccagtttagattctggggtcccatcaaggttcagtggaagtcgatctgggacagattatactttaaccatcagcagtctgcagcctgaagattttgtagtatattactgtctacaatacgccatcttcccgtacacgttcggccaagggacaaaactggagattaaagaattcggtggcggtggctcgggcggtggtgggtcgggtggcggcggatct

SEQ ID NO: 12 - protein, 276 aa, Artificial Sequence
  Description of Artificial Sequence H520C9sFv plus linker
MDMRVPAQLLGLLLLWFPGSRCEIQLVQSGPEVKKPGASVKISCKASGYTFANYGMNWMKQAPGKGLEWMGWINTYTGQSTYADDFKERFTFTLDTSTSTAHLEISSLRSEDTATYFCARRFGFAYWGQGTLVTVSSGGGGSGGGGSGGGGSDIQMTQSPSSLSASVGDRVTLTCRASQDIGNSLTWLQQKPGKTIKRLIYATSSLDSGVPSRFSGSRSGTDYTLTISSLQPEDFVVYYCLQYAIFPYTFGQGTKLEIKEFGGGGSGGGGSGGGGS

SEQ ID NO: 13 - DNA, 408 bp, Artificial Sequence
  Description of Artificial Sequence hIL-2
atgcctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttctcagagcatcatctcaacactgacttgactcgag

SEQ ID NO: 14 - protein, 133 aa, Artificial Sequence [sequence omitted]

(19) United States
(12) Patent Application Publication
(10) Pub. No.: US 2002/0041865 A1
(43) Pub. Date: Apr. 11, 2002

(54) Methods for treating tumors

(76) Inventors: Richard Austin, Ancaster (CA); Cheuk S. Kwok, Hung Hom (HK); David B. Ring, Palo Alto, CA (US)

Correspondence Address: CHIRON CORPORATION, Intellectual Property - R440, P.O. Box 8097, Emeryville, CA, 94662-8097, US

(21) Appl. No.: 09/766,543

(22) Filed: Jan. 19, 2001

Related U.S. Application Data
(60) Provisional application No. 60/177,258, filed on Jan. 20, 2000.

(51) Int. Cl. A61K 39/395; A61K 38/20

(52) U.S. Cl. 424/85.2; 424/178.1

(57) ABSTRACT

Fusions comprising IL-2 polypeptides and p185-specific binding molecules are disclosed. The fusions provide an effective means of treating p185-positive tumors in vivo.

### CROSS-REFERENCE TO RELATED APPLICATION

This application is related to provisional patent application Ser. No. 60/177,258, filed Jan. 20, 2000, from which priority is claimed under 35 USC §119(e)(1) and which is incorporated herein by reference in its entirety.

### TECHNICAL FIELD

The present invention relates generally to methods of treating tumors. In particular, the present invention pertains to interieukin-2/anti-p185 antibody fusions, useful for suppressing and/or eradicating tumor growth.

### BACKGROUND OF THE INVENTION

Recombinant human IL-2 (rhIL-2), in combination with LAK cells, has been administered to patients with advanced melanoma or renal cell carcinoma (Rosenberg S. A. J Natl Cancer Inst (1996) 96: 1635-1644). Several approaches to selectively target IL-2 to tumor sites have been employed (Sabzevari et al. Proc Natl Acad Sci USA (1994) 91:9626-9630; Becker et al. J Clin Invest (1996) 98:2801-2804; Xiang et al. Cancer Res (1997) 57:4948-4955; Goldenberg D. M. and Schlom J. Immunol Today (1993) 14:5-7; Wilder et al. J Clin Oncol (1996) 14:1383-1400; Meredith et al. Oncology (1997) 11:979-984; Sands H. Antibody Immunoconj Radiopharm (1998) 1:213:226; Kemshead J. T. and Hopkins K. J Royal Soc Med (1993) 86:219-224).

To date, several approaches of engineering conventional murine mAbs have been developed (Colcher et al. J Natl Cancer Inst (1990) 82:1191-1197; Casey et al. J Immunol Methods (1995) 179:105-116; Yokota et al. Cancer Res (1992) 52:3402-3408). A number of single-chain, antigen-binding site polypeptides and sFv molecules have been described. See, e.g., U.S. Pat. Nos. 5,132,405; 5,091,513; and 4,946,778; Chaudhary et al. Proc Natl Acad Sci USA (1990) 87:1066-1070); Reisfeld R. A. and Gillies S. D. J Clin Lab Anal (1996) 10:160-166; Chen et al. J Immunol (1994) 153:4775-4787.

Hybrid antibody molecules having variable regions derived from, for example, a murine immunoglobulin fused to constant regions derived from a human immunoglobulin have been described. See e.g., U.S. Pat. No. 4,816,567; Winter et al. (1991) Nature 349:293-299; and Lobuglio et al. (1989) Proc. Nat. Acad. Sci. USA 86:4220-4224; Dillman R. O. (1989) Ann Intern Med. 111(7):592-603. A number of recombinant or biosynthetic molecules comprising rodent antigen-binding sites have also been described. See, e.g., Riechmann et al. (1988) Nature 332:323-327; Verhoeyen et al. (1988) Science 239:1534-1536; U.K. Patent Publication No. GB 2,276,169, published Sep. 21, 1994.

Fusions of IL-2 to various tumor-specific antibodies, including fusions with whole antibodies, single-chain antibodies, and Fab fragments, have been made. (See, e.g., Chen et al. J Immunol (1994) 153:4775-4787; Reisfeld R. A. and Gillies S. D. J Clin Lab Anal (1996) 10:160-166; Savage et al. Br J Cancer (1993) 67:304-310; Xiang et al. Immunol Cell Biol (1994) 72:275-285; Gillies et al. Proc Natl Acad Sci USA (1992) 89:1428-1432; and Reisfeld R. A. and Gillies S. D. Recombinant Antibody Fusion Proteins for Cancer Immunotherapy. In: U. Gunthert et al. (eds.), Attempts to Understand Metastasis Formation III: Therapeutic Approaches for Metastasis Treatment Springer pp 27-53). However, there remains a continued need for IL-2 antibody fusions which display anti-tumor efficacy in vivo.

The human HER-2/neu (c-erbB2) proto-oncogene product, p185, is an approximately 185 kD protein (previously designated with molecular masses ranging from 185 to 210 kD) which is found in various cancerous tissues. This protein has also been designated c-erbB-2, HER2 and BCA200. The p185 protein has been described in U.S. Pat. No. 4,753,894 to Frankel et al. See, also, Frankel et al. J Biol Response Modif (1985) 4:273:286; Ring et al. Cancer Res (1989) 49:3070-3080; and Ring et al. Mol Immunol (1991) 28:915-917). This protein is a member of the tyrosinase kinase family.

Monoclonal antibodies to p185 have been made. See, e.g., U.S. Pat. Nos. 4,753,894; 5,169,774; 5,629,197; 5,677,171; 5,720,937; 5,720,934; 5,725,856; Frankel et al. J Biol Response Modif (1985) 4:273:286; Ring et al. Cancer Res (1989) 49:3070-3080; and Ring et al. Mol Immunol (1991) 28:915-917. Additionally, Carter, et al. Proc Natl Acad Sci USA 1992 89:4285-4289 describe the humanization of an anti-p185 antibody. Antibodies to p185 have been reported to have antiproliferative effects in vitro. See, e.g., Hudziak, et al. Molec Cell Biol (1989) 9:1165-1172. Doran et al. Bio/Technology (1994) 12:890-897 report the fusion of a single-chain p185 antibody, derived from murine monoclonal antibody 741F8, with IL-2.

Despite the above advances, there is a continued need for safe, new approaches to cancer therapy which provide targeted delivery of anti-tumor agents in effective amounts to the tumor microenvironment.

### DISCLOSURE OF THE INVENTION

The present invention is based on the development of humanized anti-p185 antibodies and the surprising discovery that novel fusions, which include IL-2 polypeptides linked to p185-specific binding molecules, are able to suppress tumor growth in vivo.

Accordingly, in one embodiment, the invention is directed to a method of inhibiting tumor cell growth in vivo comprising administering to a subject an effective amount of a fusion that comprises an IL-2 polypeptide linked to a p185-specific binding molecule. In certain embodiments, the p185-specific binding molecule portion of the fusion is an antibody, such as a monoclonal antibody or a humanized antibody, derived from e.g., monoclonal antibody 520C9, and the IL-2 polypeptide is native human IL-2 or a biologically active human IL-2 analog.

In other embodiments, the invention is directed to a method of inhibiting tumor cell growth in vivo comprising administering to a subject an effective amount of a fusion protein comprising the amino acid sequence depicted in FIGS. 11A through 11B (SEQ ID NO:______), or a sequence having at least about 80% sequence identity thereto.

In still further embodiments, the invention is directed to an immunoconjugate comprising a humanized anti-p185 antibody linked to an IL-2 polypeptide. In certain embodiments, the humanized anti-p185 antibody is derived from monoclonal antibody 520C9 and the IL-2 polypeptide is native, human IL-2 or a biologically active human IL-2 analog. The humanized anti-p185 antibody and the IL-2 polypeptide may be a fusion protein produced by recombinant expression of a chimeric gene encoding the fusion protein or the humanized anti-p185 antibody may chemically linked to the IL-2 polypeptide. Additionally, the C-terminus of the humanized anti-p185 antibody may be linked to the N-terminus of the IL-2 polypeptide or, conversely, the N-terminus of the humanized anti-p185 antibody is linked to the C-terminus of the IL-2 polypeptide.

In another embodiment, the subject invention is directed to an immunoconjugate comprising the amino acid sequence depicted in FIGS. 11A through 11B (SEQ ID NO:______), or a sequence having at least about 80% sequence identity thereto.

In still further embodiments, the subject invention is directed to polynucleotides encoding the above immunoconjugates, recombinant vectors comprising the polynucleotides, host cells transformed with the recombinant vectors, as well as recombinant methods of producing the immunoconjugate.

In another embodiment, the invention is directed to a method of producing an immunoconjugte comprising (a) providing a humanized anti-p185 antibody; and (b) chemically conjugating the humanized anti-p185 antibody to an IL-2 polypeptide.

In a further embodiments, the invention is directed to a humanized anti-p185 antibody comprising the amino acid sequence depicted in FIGS. 9A through 9B; a polynucleotide encoding the humanized anti-p185 antibody; a recombinant vector comprising the polynucleotide and control elements that are operably linked to the polynucleotide whereby a coding sequence within said polynucleotide can be transcribed and translated in a host cell, and at least one of the control elements is heterologous to said coding sequence; host cells transformed with the vector; and a method of producing a humanized anti-p185 antibody comprising providing a population of the transformed host cells and culturing the population of cells under conditions whereby the humanized anti-p185 antibody encoded by the coding sequence present in the recombinant vector is expressed.

These and other embodiments of the present invention will readily occur to those of ordinary skill in the art in view of the disclosure herein.

### BRIEF DESCRIPTION OF THE FIGURES

FIG. 1 is a schematic diagram of the construction of expression plasmids pcDNA-H520C9sFv-hIL-2 and pcDNA-H520C9sFv-mhIL-2.

FIG. 2 shows the results of a western blot analysis of recombinant humanized sFv antibody/IL-2 fusion proteins stably expressed in BHK cells. Lane A, 0.5 μg of rhIL-2. Lanes B, C and D, conditioned medium from BHK cells transfected with either pcDNA3.1(+), pcDNA-H520C9sFv-hIL-2 or pcDNA-H520C9sFv-mhIL-2, respectively. Lane E, H520C9sFv-hIL-2 fusion protein immunoprecipitated with anti-human IL-2 mAb, MAB202. Samples were separated on a 15% SDS-polyacrylamide gel under reducing conditions, transferred to nitrocellulose membranes and immunoblotted using the anti-human IL-2 polyclonal antibody, EP100. The fusion proteins, H520C9sFv-hIL-2 and H520C9sFv-mhIL-2 were shown to migrate as single bands of 45 kD (arrowhead).

FIG. 3 shows IL-2 activity of recombinant humanized sFv antibody/IL-2 fusion proteins. Open circles (∘) denote IL-2 activity of rhIL-2; closed circles (●) indicate IL-2 activity of conditioned medium from BHK cells transfected with pcDNA-H520C9sFv-hIL-2; triangles (▴) show IL-2 activity of conditioned medium from BHK cells transfected with pcDNA-H520C9sFv-mhIL-2. Activity was measured by [³H]-thymidine incorporation in CTLL-2 cells. The data are the mean ±S.D. from three separate experiments.

FIG. 4 shows LAK cell-mediated cytotoxicity induced by H520C9sFv-hIL-2 fusion protein. Human LAK cells (effector) were generated by incubation of fresh PBMCs with either conditioned medium from control BHK cells (cross-hatched bars), rhIL-2 (shaded, cross-hatched bars) or equivalent dose of H520C9sFv-hIL-2 (solid bars). Lysis of Daudi cells (target) was determined using a 4 hr. (Calcein AM-release assay. The data are the mean ±S.D. from three separate experiments. *,p<0.01 vs. medium from cells transfected with pcDNA 3.1(+).

FIG. 5 shows the inhibition of stimulatory effects of rhIL-2 (∘) or H520C9sFv-hIL-2 (▾) on CTLL-2 cells by the anti-human IL-2 neutralizing antibody, MAB202. Based on the molecular mass of either IL-2 or H520C9sFv-hIL-2 fusion protein, the Neutralization Dose 50 for 2 ng/ml rhIL-2 or the same dose of H520C9sFv-hIL-2 was determinated to be approximately 0.076 and 0.128 μg/ml MAB202, respectively. No inhibitory effect was observed using control mouse IgG (●). The data are the mean ±S.D. from three separate experiments.

FIG. 6 shows the antigen-binding activity of H520C9sFv-hIL-2 fusion protein. Antigen-binding activity was measured by indirect ELISA using either cultured SKOV 3ip1 or HeLa cells. Bound fusion protein was recognized with the anti-human IL-2 polyclonal antibody EP100. The fusion proteins, H520C9sFv-hIL-2 (▴) and H520C9sFv-mhIL-2 (Δ) were shown to bind to p185 positive cells SKOV 3ip1 (FIG. 6a), but not p185 negative Hela cells (FIG. 6b). A chemically-conjugated molecule containing the intact parental 520C9 mAb and rhIL-2 (∘) was used as a positive control. As a negative control, mouse IgG (●) failed to show any binding activity in both cell lines. The data are the mean ±S.D. from three separate experiments. *,p<0.01 vs. normal mouse IgG. **,p<0.05 vs. normal mouse IgG.

FIG. 7 shows that the anti-p185 mAb, 520C9, specifically blocks the binding of H520C9sFv-hIL-2 fusion protein to SKOV 3ip1 cells. SKOV 3ip1 cells, preexposed to serially diluted intact 520C9 monoclonal antibody or to mouse IgG (0.001 to 10 nM), were washed with PBS and incubated with 10 nM H520C9sFv-hIL-2 fusion protein at 37° C. for 2 h, as described below. Bound fusion protein was measured by indirect ELISA using the anti-human IL-2 polyclonal antibody, EP100. 520C9 mAb (∘) caused a dose-dependent decrease in the binding of the fusion protein to SKOV 3ip1 cells. Control mouse IgG (●) had no effect on the binding of the fusion protein. The data are the mean ±S.D. from three separate experiments. *,p<0.01 vs. normal mouse IgG.

FIG. 8 shows the amino acid sequence of native human IL-2 (SEQ ID NO:______).

FIGS. 9A through 9B depict the DNA and corresponding amino acid sequence of the 520C9 humanized single-chain antibody used in the IL-2-antibody fusions described herein (SEQ ID NO:______). The restriction sites are depicted. The underlined sequences are the complementarity determining regions (CDRs) and the bolded sequence is the artificial peptide linker connecting the antibody light and heavy chain V region sequences.

FIG. 10 depicts the effect of the H520C9sFv-hIL-2 fusion protein versus saline on tumor volume in an experimental mouse tumor model. Results are expressed as relative fold-increase in tumor volume measured 14 days post-tumor implantation. Diamonds depict results from mice injected with H520C9sFv-hIL-2 in 200 μl TBS; squares show results from mice injected with 200 μl TBS (n=4 for each group, p<0.01 after three days of treatment.

FIGS. 11A through 11B show the nucleotide sequence (SEQ ID NO:______) and corresponding amino acid sequence (SEQ ID NO:______) of H520C9sFv-hIL-2.

### DETAILED DESCRIPTION OF THE INVENTION

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990).

All publications, patents and patent applications cited herein, whether supra or infra, are hereby incorporated by reference in their entirety.

It must be noted that, as used in this specification and the appended claims, the singular forms “a”, “an” and “the” include plural referents unless the content clearly dictates otherwise. Thus, for example, reference to “a polyp eptide” includes a mixture of two or more polypeptides, and the like.

The following amino acid abbreviations are used throughout the text:

| | |
|---|---|
| Alanine: Ala (A) | Arginine: Arg (R) |
| Asparagine: Asn (N) | Aspartic acid: Asp (D) |
| Cysteine: Cys (C) | Glutamine: Gln (Q) |
| Glutamic acid: Glu (E) | Glycine: Gly (G) |
| Histidine: His (H) | Isoleucine: Ile (I) |
| Leucine: Leu (L) | Lysine: Lys (K) |
| Methionine: Met (M) | Phenylalanine: Phe (F) |
| Proline: Pro (P) | Serine: Ser (S) |
| Threonine: Thr (T) | Tryptophan: Trp (W) |
| Tyrosine: Tyr (Y) | Valine: Val (V) |

I. Definitions

In describing the present invention, the following terms will be employed, and are intended to be defined as indicated below.

The terms “polypeptide” and “protein” refer to a polymer of amino acid residues and are not limited to a minimum length of the product. Thus, peptides, oligopeptides, dimers, multimers, and the like, are included within the definition. Both full-length proteins and fragments thereof are encompassed by the definition. The terms also include postexpression modifications of the polypeptide, for example, glycosylation, acetylation, phosphorylation and the like. Furthermore, for purposes of the present invention, a “polypeptide” refers to a protein which includes modifications, such as deletions, additions and substitutions (generally conservative in nature), to the native sequence, so long as the protein maintains the desired activity. These modifications may be deliberate, as through site-directed mutagenesis, or may be accidental, such as through mutations of hosts which produce the proteins or errors due to PCR amplification. Thus, the term “IL-2 polypeptide” refers to native IL-2 sequences, as well as to IL-2 analogs, muteins and fragments, as defined further below.

By “purified” and “isolated” is meant, when referring to a polypeptide or polynucleotide, that the indicated molecule is present in the substantial absence of other biological macromolecules of the same type. The term “purified” as used herein preferably means at least 75% by weight, more preferably at least 85% by weight, more preferably still at least 95% by weight, and most preferably at least 98% by weight, of biological macromolecules of the same type are present. An “isolated polynucleotide which encodes a particular polypeptide” refers to a nucleic acid molecule which is substantially free of other nucleic acid molecules that do not encode the subject polypeptide; however, the molecule may include some additional bases or moieties which do not deleteriously affect the basic characteristics of the composition.

The term “interleukin-2” or “IL-2” as used herein refers to a compound having the primary, secondary and/or tertiary molecular structure of native IL-2, and which has IL-2 activity as measured in standard IL-2 bioassays, such as the ability to stimulate proliferation of human IL-2 dependent cytolytic and helper T-cell lines (see, Gillis et al., J. Immunol. (1978) 120:2027-2032; Watson, J., J. exp. Med. (1979) 1570:1510-1519). The IL-2 molecule may include posttranslational modifications, such as glycosylation, acetylation, phosphorylation, etc. Furthermore, as ionizable amino and carboxyl groups are present in the molecule, a particular IL-2 may be obtained as an acidic or basic salt, or in neutral form.

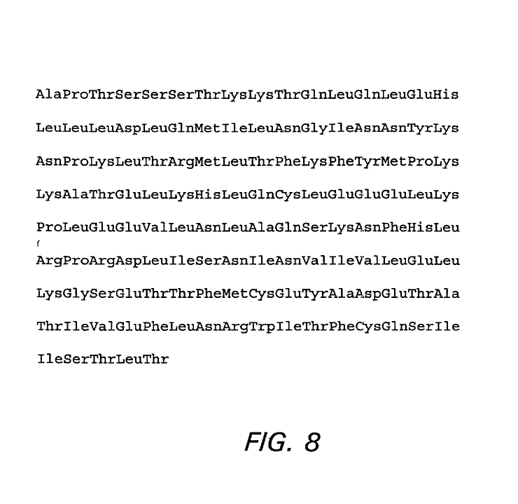

Additionally, for purposes of the present invention, an IL-2 polypeptide may be derived from any of several tissues of any mammalian source, such as human, bovine, murine, canine, equine, ovine, porcine, etc. The sequences for various IL-2 proteins are known. For example, the amino acid sequence of native, human IL-2 is shown in FIG. 8 (SEQ ID NO:______) and bovine IL-2 is described in Cerretti et al., Proc. Natl. Acad. Sci USA (1986) 83:3223-3227. The IL-2 compound may be purified directly from the source organism, or may be recombinantly or synthetically produced (see further below).

The terms “IL-2 analog” and “IL-2 mutein” refer to biologically active derivatives of IL-2, or fragments of such derivatives, that retain IL-2 activity, as measured in assays as described above. In general, the term “analog” refers to compounds having a native polypeptide sequence and structure with one or more amino acid additions, substitutions (generally conservative in nature) and/or deletions, relative to the native molecule, so long as the modifications do not destroy IL-2 activity. The term “mutein” refers to peptides having one or more peptide mimics (“peptoids”), such as those described in International Publication No. WO 91/04282. Preferably, the analog or mutein has at least the same activity as the native molecule. Methods for making polypeptide analogs and muteins are known in the art and are described further below.

Particularly preferred analogs include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalaninc, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity.

By “fragment” is intended a polypeptide consisting of only a part of the intact polypeptide sequence and structure. The fragment can include a C-terminal deletion or N-terminal deletion of the native polypeptide. A “fragment” of IL-2 will generally include at least about 10 contiguous amino acid residues of the full-length molecule, preferably at least about 15-25 contiguous amino acid residues of the full-length molecule, and most preferably at least about 20-50 or more contiguous amino acid residues of full-length IL-2, or any integer between 10 amino acids and the full-length sequence, provided that the fragment in question retains IL-2 activity as described above. One preferred fragment of IL-2 is a molecule having one or more of the first five N-terminal amino acids of the native IL-2 molecule deleted.

By “recombinant IL-2” is intended an IL-2 molecule having biological activity, as measured using the techniques described above and which has been prepared by recombinant DNA techniques as described herein. See, also, for example, Taniguchi, et al. Nature (1983) 302:305-310 and Devos Nucleic Acids Research (1983) 11:4307-4323 or mutationally altered IL-2 as described by Wang, et al. Science (1984) 224:1431-1433. In general, the gene coding for IL-2 is cloned and then expressed in transformed organisms, as described further below. The host organism expresses the foreign gene to produce IL-2 under expression conditions. Processes for growing, harvesting, disrupting, or extracting the IL-2 from cells are substantially described in, for example, U.S. Pat. Nos. 4,604,377; 4,738,927; 4,656,132; 4,569,790; 4,748,234; 4,530,787; 4,572,298; and 4,931,543, herein incorporated by reference in their entireties.

By “p185” is meant the protein product of the human HER-2/neu (c-erbB2) gene, described in e.g., U.S. Pat. No. 4,753,894 to Frankel et al.; Frankel et al. J Biol Response Modif (1985) 4:273:286; Ring et al. Cancer Res (1989) 49:3070-3080; and Ring et al. Mol Immunol (1991)28:915-917. This protein, variously known as p185, c-erbB-2, HER2 and BCA200, has a molecular mass of approximately 185 kD (previously designated with molecular masses ranging from 185 to 210 kD) and is found in various cancerous tissues.

A “specific binding molecule” intends a molecule that, through chemical or physical means, specifically binds to a second molecule. Thus, a p185-specific binding molecule is a molecule that specifically binds p185. One example of a specific binding molecule is an antibody molecule. The term “antibody” as used herein includes antibodies obtained from both polyclonal and monoclonal preparations, as well as, the following: hybrid (chimeric) antibody molecules (see, for example, Winter et al. (1991) Nature 349:293-299; and U.S. Pat. No. 4,816,567); F(ab′)₂ and F(ab) fragments; Fv molecules (non-covalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, for example, Huston et al. (1988) Proc Natl Acad Sci USA 85:5879-5883); dimeric and trimeric antibody fragment constructs; minibodies (see, e.g., Pack et al. (1992) Biochem 31:1579-1584; Cumber et al. (1992) J Immunology 149B: 120-126); humanized antibody molecules (see, for example, Riechmann et al. (1988) Nature 332:323-327; Verhoeyan et al. (1988) Science 239:1534-1536; and U.K. Patent Publication No. GB 2,276,169, published Sep. 21, 1994); and, any functional fragments obtained from such molecules, wherein such fragments retain immunological binding properties of the parent antibody molecule.

As used herein, the term “monoclonal antibody” refers to an antibody composition having a homogeneous antibody population. The term is not limited regarding the species or source of the antibody, nor is it intended to be limited by the manner in which it is made. Thus, the term encompasses antibodies obtained from murine hybridomas, as well as human monoclonal antibodies obtained using human rather than murine hybridomas. See, e.g., Cote, et al. Monclonal Antibodies and Cancer Therapy, Alan R. Liss, 1985, p. 77.

An IL-2 polypeptide is “linked” to a p185-specific binding molecule when the IL-2 polypeptide is chemically coupled to, or associated with the specific binding molecule, or when expressed from a chimeric polynucleotide which encodes the IL-2 polypeptide and the p185-specific binding molecule of interest. The term “linked” intends that the IL-2 polypeptide may either be directly linked to the p185-specific binding molecule or may be linked via a linker moiety, such as via a peptide linker described below.

An “immunoconjugate” or “fusion” is an IL-2 polypeptide which is linked to a p185-specific binding molecule, as defined above. Thus, the term denotes both chemically conjugated as well as recombinantly produced fusion molecules.

The terms “recombinant DNA molecule,” or “recombinant polynucleotide” are used herein to refer to a polynucleotide of genomic, cDNA, semisynthetic, or synthetic origin which, by virtue of its origin or manipulation: (1) is not associated with all or a portion of a polynucleotide with which it is associated in nature, (2) is linked to a polynucleotide other than that to which it is linked in nature, or (3) does not occur in nature. Thus, the term encompasses “synthetically derived” nucleic acid molecules.

The term “polynucleotide” or “nucleic acid molecule” as used herein refers to a polymeric form of nucleotides of any length, either ribonucleotides or deoxyribonucleotides. This term refers only to the primary structure of the molecule and thus includes double- and single-stranded DNA and RNA. It also includes known types of modifications, for example, labels which are known in the art, methylation, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example proteins (including for e.g., nucleases, toxins, antibodies, signal peptides, poly-L-lysine, etc.), those with intercalators (e.g., acridine, psoralen, etc.), those containing chelates (e.g., metals, radioactive metals, boron, oxidative metals, etc.), those containing alkylators, those with modified linkages (e.g., alpha anomeric nucleic acids, etc.), as well as unmodified forms of the polynucleotide.

A “coding sequence” is a nucleic acid molecule which is translated into a polypeptide, usually via mRNA, when placed under the control of appropriate regulatory sequences. The boundaries of the coding sequence may be determined by a translation start codon at the 5′-terminus and a translation stop codon at the 3′-terminus. A coding sequence can include, but is not limited to, cDNA, and recombinant nucleotide sequences.

“Control sequences” refer to nucleic acid sequences which are necessary to effect the expression of coding sequences to which they are ligated. The nature of such control sequences differs depending upon the host organism; in prokaryotes, such control sequences generally include promoter, ribosomal binding site, and transcription termination sequence; in eukaryotes, generally, such control sequences include promoters and transcription termination sequences. The term “control sequences” is intended to include, at a minimum, all components necessary for expression of a coding sequence, and may also include additional components, for example, leader sequences and fusion partner sequences.

A control element, such as a promoter, “directs the transcription” of a coding sequence in a cell when RNA polymerase will bind the promoter and transcribe the coding sequence into mRNA, which is then translated into the polypeptide encoded by the coding sequence.

“Operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

As used herein, the term “expression cassette” refers to a molecule comprising at least one coding sequence operably linked to a control sequence which includes all nucleotide sequences required for the transcription of cloned copies of the coding sequence and the translation of the mRNAs in an appropriate host cell. Such expression cassettes can be used to express eukaryotic genes in a variety of hosts such as bacteria, blue-green algae, plant cells, yeast cells, insect cells and animal cells. Under the invention, expression cassettes can include, but are not limited to, cloning vectors, specifically designed plasmids, viruses or virus particles. The cassettes may further include an origin of replication for autonomous replication in host cells, selectable markers, various restriction sites, a potential for high copy number and strong promoters.

By “vector” is meant any genetic element, such as a plasmid, phage, transposon, cosmid, chromosome, virus etc., which is capable of replication when associated with the proper control elements and which can transfer gene sequences between cells. Thus, the term includes cloning and expression vehicles, as well as viral vectors.

A cell has been “transformed” by an exogenous polynucleotide when the polynucleotide has been introduced inside the cell membrane. The exogenous polynucleotide may or may not be integrated (covalently linked) into chromosomal DNA making up the genome of the cell. In procaryotes and yeasts, for example, the exogenous DNA may be maintained on an episomal element, such as a plasmid. With respect to eucaryotic cells, a stably transformed cell is one in which the exogenous DNA has become integrated into the chromosome so that it is inherited by daughter cells through chromosome replication. This stability is demonstrated by the ability of the eucaryotic cell to establish cell lines or clones comprised of a population of daughter cells containing the exogenous DNA.

A “host cell” is a cell which has been transformed, or is capable of transformation, by an exogenous nucleic acid molecule.

As used herein, the terms “treating” or “treatment” of a disease include preventing the disease, i.e. preventing clinical symptoms of the disease in a subject that may be exposed to, or predisposed to, the disease, but does not yet experience or display symptoms of the disease; inhibiting the disease, i.e., arresting the development of the disease or its clinical symptoms, such as by suppressing tumor cell growth; or relieving the disease, i.e., causing regression of the disease or its clinical symptoms.

The terms “effective amount” or “pharmaceutically effective amount” refer to a nontoxic but sufficient amount of the agent to provide the desired biological result. That result can be reduction and/or alleviation of the signs, symptoms, or causes of a disease, such as cancer, or any other desired alteration of a biological system. Such amounts are described below. An appropriate “effective” amount in any individual case may be determined by one of ordinary skill in the art using routine experimentation.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

By “physiological pH” or a “pH in the physiological range” is meant a pH in the range of approximately 7.2 to 8.0 inclusive, more typically in the range of approximately 7.2 to 7.6 inclusive.

As used herein, the term “subject” encompasses mammals and non-mammals. Examples of mammals include, but are not limited to, any member of the Mammalia class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or sex.

II. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of compositions and methods similar or equivalent to those described herein can be used in the practice of the present invention, the preferred materials and methods are described herein.

The present invention is based on the development of a novel, humanized anti-p185 antibody and the discovery that fusions comprising IL-2 polypeptides, linked to p185-specific binding molecules, provide a safe and effective way of treating cancer in a subject suffering therefrom. The result is surprising because high-dose IL-2 therapy is known to cause severe systemic toxicity in normal tissues. Accordingly, its clinical use has heretofore been limited.

The molecules of the present invention are therefore useful in the therapy of malignant and/or benign tumors which express the p185 protein, including, without limitation, breast, renal, gastric, gastrointestinal and salivary gland tumors, and a variety of other human adenocarcinomas.

The method uses conjugates of IL-2 polypeptides with p185-specific binding molecules. In preferred embodiments, the p185-specific binding molecule is an antibody and the molecules of the present invention are fusion proteins expressed recombinantly from a chimeric polynucleotide encoding the IL-2-anti-p185 antibody immunoconjugate. However, the components of the immunoconjugates may also be produced individually and subsequently linked via chemical conjugation techniques.

The IL-2 portion of the subject molecules may be linked to either the C- or N-terminus of the p185-specific binding portion. Additionally, a linker moiety may be present between the IL-2 and p185-specific binding portions of the molecule. Such linkers are generally formed from small hydrophilic amino acid residues that do not tend to coil or form secondary structures. The linkers are therefore preferably free of amino acid residues having large side groups which might interfere with proper folding of the antibody portion of the molecule. See, e.g., U.S. Pat. Nos. 5,091,513 and 5,132,405 to Huston et al.; and U.S. Pat. No. 4,946,778 to Ladner et al. Thus, suitable linkers under the invention generally comprise polypeptide chains of glycine and/or serine residues, such as polyglycine peptides or alternating sets of glycine and serine residues, and may include glutamic acid and lysine residues to enhance solubility. Nonlimiting examples of particular linkers for use with the present fusions include a linker which has the amino acid sequence [(Gly)₄Ser]₃, and a linker comprising 2 or 3 repeats of [(Ser)₄Gly], such as [(Ser)₄Gly]₃. Nucleotide sequences encoding such linker moieties can be readily provided using various oligonucleotide synthesis techniques known in the art (see, e.g., Sambrook, supra) and placed on either end of either of the molecules, depending on the orientation of the final fusion desired.

IL-2 Polypeptides

The IL-2 portion of the subject molecules may be a native IL-2 sequence such as native, human IL-2, as depicted in FIG. 8 (SEQ ID NO:______), or a biologically active analog, mutein or fragment of IL-2, as defined above, that retains IL-2 activity, as measured in standard IL-2 bioassays, such as the ability to stimulate proliferation of human IL-2 dependent cytolytic and helper T-cell lines (see, Gillis et al., J. Immunol. (1978) 120:2027-2032; Watson, J., J. exp. Med. (1979) 1570:1510-1519). The art provides substantial guidance regarding the preparation and use of such molecules.

For example, several IL-2 analogs are known in the art and include those described in e.g., European Patent Publication Nos. 136,489, 91,539, 88,195, 109,748; U.S. Pat. Nos. 4,518,584, 4,588,584, 4,752,585, 4,931,543, 5,206,344; all of which are incorporated herein by reference. Particular analogs include, for example, an IL-2 molecule that lacks the initial N-terminal alanine of the native molecule and wherein the cysteine normally present at position 125 is substituted with a neutral amino acid such as serine or alanine (des-ala-1, ser-125 IL-2 or des-ala-1, ala-125 IL-2) (see, U.S. Pat. Nos. 4,518,584 and 4,588,584) or where the methionine at position 104 is replaced with a neutral amino acid such as alanine (des-ala-1, ala-104), as well as molecules retaining the N-terminal alanine and having the above substitutions. These and other analogs which retain IL-2 activity will find use with the methods described herein.

IL-2 analogs will generally have at least 60%, pre lerably 70%, more preferably 80%, preferably 90% to 95% or more, and most preferably 98% or more, amino acid sequence identity to the amino acid sequence of the reference IL-2 molecule. For example, the IL-2 analog may have from about 1 to about 50 amino acid substitutions, or any integer in between, e.g., 25,20, 15, 10, 9, 8, 7, 6, 5, 4, 3, 2 or 1 amino acid substitutions. By “sequence identity” is intended the same amino acid residues are found within the variant polypeptide and the polypeptide molecule that serves as a reference when a specified, contiguous segment of the amino acid sequence of the variant is aligned and compared to the amino acid sequence of the reference molecule. The percentage sequence identity between two amino acid sequences is calculated by determining the number of positions at which the identical amino acid residue occurs in both sequences to yield the number of matched positions, dividing the number of matched positions by the total number of positions in the segment undergoing comparison to the reference molecule, and multiplying the result by 100 to yield the percentage of sequence identity.

For purposes of optimal alignment of the two sequences, the contiguous segment of the amino acid sequence of the analog may have additional amino acid residues or deleted amino acid residues with respect to the amino acid sequence of the reference molecule. The contiguous segment used for comparison to the reference amino acid sequence will comprise at least twenty (20) contiguous amino acid residues, and may be 30, 40, 50, 100, or more residues. Corrections for increased sequence identity associated with inclusion of gaps in the analog's amino acid sequence can be made by assigning gap penalties. Methods of sequence alignment are well known in the art for both amino acid sequences and for the nucleotide sequences encoding amino acid sequences.

Thus, the determination of percent identity between any two sequences can be accomplished using a mathematical algorithm. One preferred, non-limiting example of a mathematical algorithm utilized for the comparison of sequences is the algorithm of Myers and Miller CABIOS (1988) 4:11-17. Such an algorithm is utilized in the ALIGN program (version 2.0), which is part of the GCG sequence alignment software package. A PAM120 weight residue table, a gap length penalty of 12., and a gap penalty of 4 can be used with the ALIGN program when comparing amino acid sequences. Another preferred, nonlimiting example of a mathematical algorithm for use in comparing two sequences is the algorithm of Karlin and Altschul Proc. Natl. Acad. Sci. USA (1990) 87:2264, modified as in Karlin and Altschul Proc. Natl. Acad. Sci. USA (1993) 90:5873-5877. Such an algorithm is incorporated into the NBLAST and XBLAST programs of Altschul et al. J. Mol. Biol. (1990) 215:403. BLAST nucleotide searches can be performed with the NBLAST program, score=100, wordlength=12, to obtain nucleotide sequences homologous to a nucleotide sequence encoding the polypeptide of interest. BLAST protein searches can be performed with the XBLAST program, score=50, wordlength=3, to obtain amino acid sequences homologous to the polypeptide of interest. To obtain gapped alignments for comparison purposes, Gapped BLAST can be utilized as described in Altschul et al. Nucleic Acids Res. (1997) 25:3389. Alternatively, PSI-Blast can be used to perform an iterated search that detects distant relationships between molecules. See, Altschul et al. (1997) supra. When utilizing BLAST, Gapped BLAST, and PSI-Blast programs, the default parameters of the respective programs (e.g., XBLAST and NBLAST) can be used. See, http://www.ncbi.nlm.nih.gov. Also see, the ALIGN program (Dayhoff (1978) in Atlas of Protein Sequence and Structure 5: Suppl. 3 (National Biomedical Research Foundation, Washington, D.C.) and programs in the Wisconsin Sequence Analysis Package, Version 8 (available from Genetics Computer Group, Madison, Wis.), for example, the GAP program, where default parameters of the programs are utilized.

When considering percentage of amino acid sequence identity, some amino acid residue positions may differ as a result of conservative amino acid substitutions, which do not affect properties of protein function. In these instances, percent sequence identity may be adjusted upwards to account for the similarity in conservatively substituted amino acids. Such adjustments are well known in the art. See, for example, Myers and Miller Computer Applic. Biol. Sci. (1988) 4:11-17.

As is readily apparent, one of skill in the art can determine the proper search parameters to use for a given sequence in the above programs. For example, the search parameters may vary based on the size of the sequence in question. Thus, for example, a representative embodiment of the present invention would include an IL-2 polypeptide having X contiguous amino acids, wherein (i) the X contiguous amino acids have at least about 50% identity to Y contiguous amino acids of the native, human IL-2 sequence, (ii) X equals Y, and (iii) X is greater than or equal to 10 amino acids and up to 250 amino acids, preferably greater than or equal to 20 amino acids and up to 250 amino acids, more preferably 30-50 amino acids and up to 250 amino acids, and even more preferably 40-75 amino acids, up to 133 amino acids, including all integer values falling between the above-described ranges.

As explained above, the subject fusions between IL-2 and the p185-specific binding molecule are generally accomplished recombinantly, by expression of a chimeric polynucleotide encoding the same. In this embodiment, a polynucleotide encoding the desired IL-2 polypeptide is linked to a polynucleotide encoding the p185-specific binding molecule, optionally using a linker moiety as described above.

Methods of obtaining IL-2-encoding polynucleotides are known in the art and described in, e.g., U.S. Pat. No. 5,206,344, Taniguchi, et al. Nature (1983) 302:305-310; Devos Nucleic Acids Research (1983) 11:4307-4323; and Wang, et al. Science (1984) 224:1431-1433.

However, the IL-2 portion of the molecule may also be provided separately and subsequently conjugated to the p185-specific binding portion. In this embodiment, the IL-2 polypeptide can also be recombinantly produced, or can be isolated directly from a tissue or organ that produces the same. Procedures for purifying native IL-2 from T-cells are described by Watson et al. J. Exp. Med. (1979) 150:849-861; Gillis et al. J. Immunology (1980) 124:1954-1962; Mochizuki et al. J. Immun. Meth. (1980) 39:185-201; Welte et al. J. Exp. Med. (1982) 156:454-464; and European patent applications 83103582.9 (published Oct. 26, 1983 under no. 92163) and 83400938.3 (published Nov. 16, 1983 under no. 94317). In general these procedures involve precipitating proteins from culture supernatants with ammonium sulfate followed by a chromatographic fractionation.

Alternatively, polypeptides for use in the subject methods can be synthesized chemically, by any of several techniques that are known to those skilled in the peptide art. See, e.g., J. M. Stewart and J. D. Young, Solid Phase Peptide Synthesis (Pierce Chemical Co., Rockford, IL 1984) and G. Barany and R. B. Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis, Synthesis, Biology, Vol. 1, for classical solution synthesis. The polypeptides of the present invention can also be chemically prepared by the method of simultaneous multiple peptide synthesis. See, e.g., Houghten Proc. Natl. Acad. Sci. USA (1985) 82:5131-5135; U.S. Pat. No. 4,631,211.

p185-specific Binding Molecules

The p185-specific binding portion of the subject molecules may be derived from polyclonal or monoclonal antibody preparations, may be a human antibody, or may be a hybrid or chimeric antibody, such as a humanized antibody, an altered antibody, F(ab′)₂ fragments, F(ab) fragments, Fv fragments, a single-domain antibody, a dimeric or trimeric antibody fragment construct, a minibody, or functional fragments thereof which bind to tumor cells expressing p185.

For purposes of the following discussion, the “antigen-binding site,” or “binding portion” of an antibody refers to the part of the immunoglobulin molecule that participates in antigen binding. The antigen binding site is typically formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs”. Thus the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Antibodies for use with the present invention can be produced using techniques well established in the art. For example, polyclonal antibodies are generated by immunizing a suitable animal, such as a mouse, rat, rabbit, sheep or goat, with an antigen of interest. In order to enhance immunogenicity, the antigen can be linked to a carrier prior to immunization. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Furthermore, the antigen may be conjugated to a bacterial toxoid, such as toxoid from diphtheria, tetanus, cholera, etc., in order to enhance the immunogenicity thereof.

Rabbits, sheep and goats are preferred for the preparation of polyclonal sera when large volumes of sera are desired. These animals are good design choices also because of the availability of labeled anti-rabbit, anti-sheep and anti-goat antibodies. Immunization is generally performed by mixing or emulsifying the antigen in saline, preferably in an adjuvant such as Freund's complete adjuvant, and injecting the mixture or emulsion parenterally (generally subcutaneously or intramuscularly). The animal is generally boosted 2-6 weeks later with one or more injections of the antigen in saline, preferably using Freund's incomplete adjuvant. Antibodies may also be generated by in vitro immunization, using methods known in the art. Polyclonal antiserum is then obtained from the immunized animal.

Monoclonal antibodies are generally prepared using the method of Kohler and Milstein (1975) Nature 256:495-497, or a modification thereof. Typically, a mouse or rat is immunized as described above. However, rather than bleeding the animal to extract serum, the spleen (and optionally several large lymph nodes) is removed and dissociated into single cells. If desired, the spleen cells may be screened (after removal of nonspecifically adherent cells) by applying a cell suspension to a plate or well coated with the antigen. B-cells, expressing membrane-bound immunoglobulin specific for the antigen, will bind to the plate, and are not rinsed away with the rest of the suspension. Resulting B-cells, or all dissociated spleen cells, are then induced to fuse with myeloma cells to form hybridomas, and are cultured in a selective medium (e.g., hypoxanthine, aminopterin, thymidine medium, “HAT”). The resulting hybridomas are plated by limiting dilution, and are assayed for the production of antibodies which bind specifically to the immunizing antigen (and which do not bind to unrelated antigens). The selected monoclonal antibody-secreting hybridomas are then cultured either in vitro (e.g., in tissue culture bottles or hollow fiber reactors), or in vivo (e.g., as ascites in mice).

Human monoclonal antibodies will also find use with the present invention and are obtained using human rather than murine hybridomas. See, e.g., Cote, et al. Monclonal Antibodies and Cancer Therapy, Alan R. Liss, 1985, p. 77.

Monoclonal antibodies or portions thereof may be identified by first screening a B-cell cDNA library for DNA molecules that encode antibodies that specifically bind to p185, according to the method generally set forth by Huse et al. (1989) Science 246:1275-1281. The DNA molecule may then be cloned and amplified to obtain sequences that encode the antibody (or binding domain) of the desired specificity.

As explained above, antibody fragments which retain the ability to recognize p185-expressing tumor cells, will also find use in the subject conjugates. A number of antibody fragments are known in the art which comprise antigen-binding sites capable of exhibiting immunological binding properties of an intact antibody molecule. For example, functional antibody fragments can be produced by cleaving a constant region, not responsible for antigen binding, from the antibody molecule, using e.g., pepsin, to produce F(ab′)₂ fragments. These fragments will contain two antigen binding sites, but lack a portion of the constant region from each of the heavy chains. Similarly, if desired, Fab fragments, comprising a single antigen binding site, can be produced, e.g., by digestion of polyclonal or monoclonal antibodies with papain. Functional fragments, including only the variable regions of the heavy and light chains, can also be produced, using standard techniques such as recombinant production or preferential proteolytic cleavage of immunoglobulin molecules. These fragments are known as F_{V}. See, e.g., Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

A single-chain Fv (“sFv” or “scFv”) polypeptide is a covalently linked V_{H}-V_{L} heterodimer which is expressed from a gene fusion including V_{H}- and V_{L}-encoding genes linked by a peptide-encoding linker. Huston et al. (1988) Proc. Nat. Acad. Sci. USA 85:5879-5883. A number of methods have been described to discern and develop chemical structures (linkers) for converting the naturally aggregated, but chemically separated, light and heavy polypeptide chains from an antibody V region into an sFv molecule which will fold into a three dimensional structure substantially similar to the structure of an antigen-binding site. See, e.g., U.S. Pat. Nos. 5,091,513, 5,132,405 and 4,946,778. The sFv molecules may be produced using methods described in the art. See, e.g., Huston et al. (1988) Proc. Nat. Acad. Sci. USA 85:5879-5883; U.S. Pat. Nos. 5,091,513, 5,132,405 and 4,946,778. Design criteria include determining the appropriate length to span the distance between the C-terminus of one chain and the N-terminus of the other, wherein the linker is generally formed from small hydrophilic amino acid residues that do not tend to coil or form secondary structures. Such methods have been described in the art. See, e.g., U.S. Pat. Nos. 5,091,513, 5,132,405 and 4,946,778. Suitable linkers generally comprise polypeptide chains of alternating sets of glycine and serine residues, and may include glutamic acid and lysine residues inserted to enhance solubility.

One method of obtaining nucleotide sequences encoding sFv molecules is by an overlap PCR approach. See, e.g., Horton et al. (1990) BioTechniques 8:528-535. The ends of the light and heavy chain variable regions that are to be joined through a linker sequence are first extended by PCR amplification of each variable region, using primers that contain the terminal sequence of the variable region followed by all or most of the desired linker sequence. After this extension step, the light and heavy chain variable regions contain overlapping extensions which jointly contain the entire linker sequence, and which can be annealed at the overlap and extended by PCR to obtain the complete sFv sequence using methods known in the art.

“Mini-antibodies” or “minibodies” will also find use with the present invention. Minibodies are sFv polypeptide chains which include oligomerization domains at their C-termini, separated from the sFv by a hinge region. Pack et al. (1992) Biochem 31:1579-1584. The oligomerization domain comprises self-associating α-helices, e.g., leucine zippers, that can be further stabilized by additional disulfide bonds. The oligomerization domain is designed to be compatible with vectorial folding across a membrane, a process thought to facilitate in vivo folding of the polypeptide into a functional binding protein.

Generally, minibodies are produced using recombinant methods well known in the art. See, e.g., Pack et al. (1992) Biochem 31:1579-1584; Cumber et al. (1992) J Immunology 149B: 120-126; and International application Nos. PCT/US92/07986, published Apr. 1, 1993, and PCT/US92/10140, published Jun. 10, 1993. For example, International application PCT/US92/07986 describes methods for making bifunctional F(ab′)₂ molecules composed of two F(ab′) monomers linked through cysteine amino acids located at the C-terminus of the first constant domain of each heavy chain. International application PCT/US92/10140 also discloses bifunctional F(ab′)₂ dimers which, in addition to the cysteine residues located in the hinge region, also contain C-terminal leucine zipper domains that further stabilize the F(ab′)₂ dimers. In both cases, the resulting F(ab′)₂ dimers are ≧100 kD in size, and thus smaller than intact immunoglobulins. The generation of (FvCys)₂ heterodimers by chemically crosslinking two V_{H}.cys domains together is described by Cumber et al. (1992) J Immunology 149B: 120-126.

Chimeric antibody molecules will also find use with the present invention. A chimeric antibody can include antigen-binding sites, such as variable regions, or fragments of variable regions, derived from a non-human immunoglobulin, which retain specificity for p185. The remainder of the antibody can be derived from the species in which the antibody will be used. Thus, if the antibody is to be used in a human, the antibody can be “humanized” in order to reduce immunogenicity yet retain activity. Such chimeric antibodies may contain not only combining sites for p185, but also binding sites for other proteins. In this way, bifunctional reagents can be generated with targeted specificity to, e.g., both external and internal antigens. For a description of chimeric antibodies and methods of generating the same, see, the examples below, as well as, e.g., Winter et al. (1991) Nature 349:293-299; Lobuglio et al. (1989) Proc. Nat. Acad. Sci. USA 86:4220-4224; Shaw et al. (1987) J Immunol 138:4534-4538; and Brown et al. (1987) Cancer Res 47:3577-3583) (each describing chimeric antibodies comprising rodent V regions and associated CDRs fused to human constant domains); Jones et al. (1986) Nature 321:522-525; Riechmann et al. (1988) Nature 332:323-327; and Verhoeyen et al. (1988) Science 239:1534-1536 (each describing rodent CDRs grafted into a human supporting FR prior to fusion with an appropriate human antibody constant domain); European Patent Publication No. 519,596, published Dec. 23, 1992 (describing rodent CDRs supported by recombinantly veneered rodent FRs).

Antibodies with veneered FRs can be produced as follows. Initially, the FR sequences derived from the V_{H} and V_{L} domains of an antibody molecule produced by hybridoma cell lines are compared with corresponding FR sequences of human variable domains obtained from an appropriate database (see, e.g., Kabat et al., in Sequences of Proteins of Immunological Interest, 4th ed., (U.S. Dept. of Health and Human Services, U.S. Government Printing Office, 1987)) and updates to the database. Human frameworks with a high degree of sequence similarity to those of the murine regions are identified. Sequence similarity is measured using identical residues as well as evolutionarily conservative amino acid substitutions. Similarity searches are performed using the selected murine framework sequence from which the CDRs have been removed. The framework sequence is used to query a database of human immunoglobulin sequences derived from multiple sources. Sequences with a high degree of sequence similarity are examined individually for their potential as humanizing framework sequences. In this way, the human homolog providing the CDRs from selected molecules with the structure most similar to their native murine framework is selected as the template for the construction of the veneered FRs.

The selected human V regions are then compared residue-by-residue to the corresponding murine amino acids. The residues in the murine FRs which differ from the selected human counterpart are replaced by the residues present in the human moiety using recombinant techniques well known in the art. Residue switching is only carried out with moieties which are at least partially exposed (solvent accessible), and care is exercised in the replacement of amino acid residues which may have a significant effect on the tertiary structure of V region domains, such as proline, glycine and charged amino acids.

In this manner, the resultant “veneered” FRs are designed to retain the murine CDR residues, the residues substantially adjacent to the CDRs, the residues identified as buried or mostly buried (solvent inaccessible), the residues believed to participate in noncovalent interchain interactions, and the residues from conserved structural regions of the FRs which are believed to influence the “canonical” tertiary structures of the CDR loops. Expression vectors including the recombinant nucleotide sequences encoding these molecules can be introduced into suitable host cells for the expression of recombinant human antibodies which exhibit the antigen specificity of the murine antibody molecule. Additionally, coexpression of complementary V_{H} and V_{L} molecules having veneered frameworks provides a convenient method of producing a heterodimeric polypeptide, featuring an antigen-binding site that binds specifically to p185, and which is weakly-immunogenic, or substantially non-immunogenic in a human recipient. For a further description of the veneering process see, e.g., European Patent Publication No. 519,596 and International Publication No. WO 92/22653.

The nucleic acid sequence and the predicted amino acid sequence of the V_{H} and V_{L} encoding domains, including the sequences of the CDRs and FRs, of a representative anti-p128 monoclonal antibody, 520C9, are described in U.S. Pat. No. 6,054,561, incorporated herein by reference in its entirety.

Each of the synthetic molecules described herein may be expressed using a monovalent phage display system as described by Garrad et al. (1991) Biotechnology 9:1373-1377, to identify V_{H}/V_{L} pairs with a desired specificity or to modify the specificity or affinity of a given V_{H}/V_{L} pair. In this regard, improvement of antigen binding affinity of a given V_{H}/V_{L} pair can be accomplished by constructing phage libraries—in which at least one CDR comprises a synthetic or point mutated CDR—and screening the phage system for molecules which exhibit enhanced binding characteristics or lower off rates using ligand affinity analysis methods well known in the art. Additionally, a phage display system can be used under the invention to facilitate “chain shuffling” in which a given V_{H} or V_{L} is re-paired with a library of random V_{L} or V_{H} sequences and the resulting phage screened for desired antigen binding behavior. Suitable phage display systems have been described (McCafferty et al. (1990) Nature 348:552-554), and chain shuffling techniques are known in the art. See, e.g., Figini et al. (1994) J. Mol. Biol. 239:68-78.

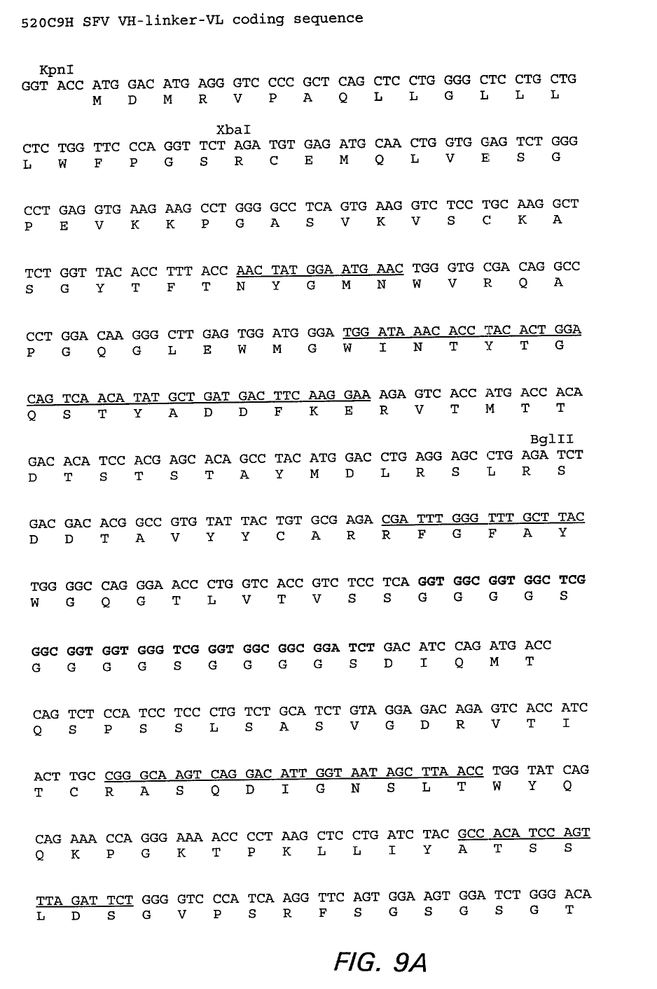

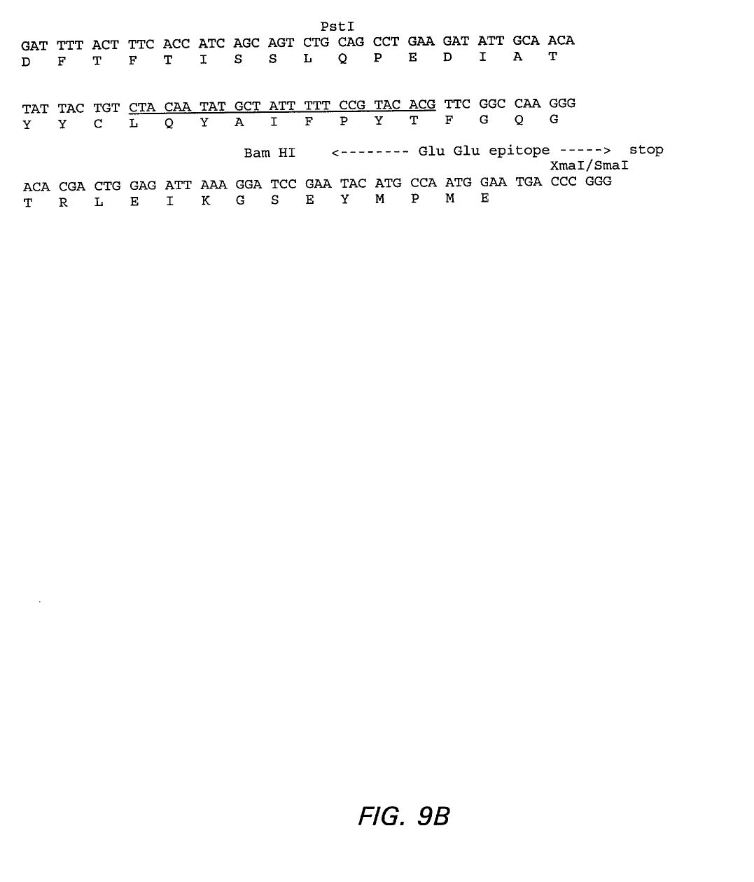

Examples of particular anti-p185 antibodies useful in the present invention include, but are not limited to, antibody molecules such as monoclonal antibodies, hybrid or chimeric antibodies, such as humanized antibodies, altered antibodies, F(ab′)₂ fragments, F(ab) fragments, Fv fragments, single-domain antibodies, dimeric or trimeric antibody fragment constructs, minibodies, or functional fragments thereof, derived from monoclonal antibodies 520C9, 454C11, 452F2, 736G9, 741F8, 758G5 and 761B10 (described in U.S. Pat. Nos. 4,753,894; 5,169,774; and 5,629,197), as well as antibody molecules derived from monoclonal antibodies 4D5, 3E8 and 3H4 (described in U.S. Pat. Nos. 5,677,171; 5,720,937; 5,720,934; 5,725,856; 5,770,195; and 5,772,997). Particularly preferred is the humanized, single-chain antibody with the sequence depicted in FIGS. 9A through 9B, derived from monoclonal antibody 520C9.

IL-2/p185-Specific Binding Molecule Fusions

Once the antibodies or polynucleotides encoding the same are produced, they are bound to the IL-2 polypeptide or polynucleotide encoding the IL-2 polypeptide, respectively, to form the immunoconjugates of the invention.

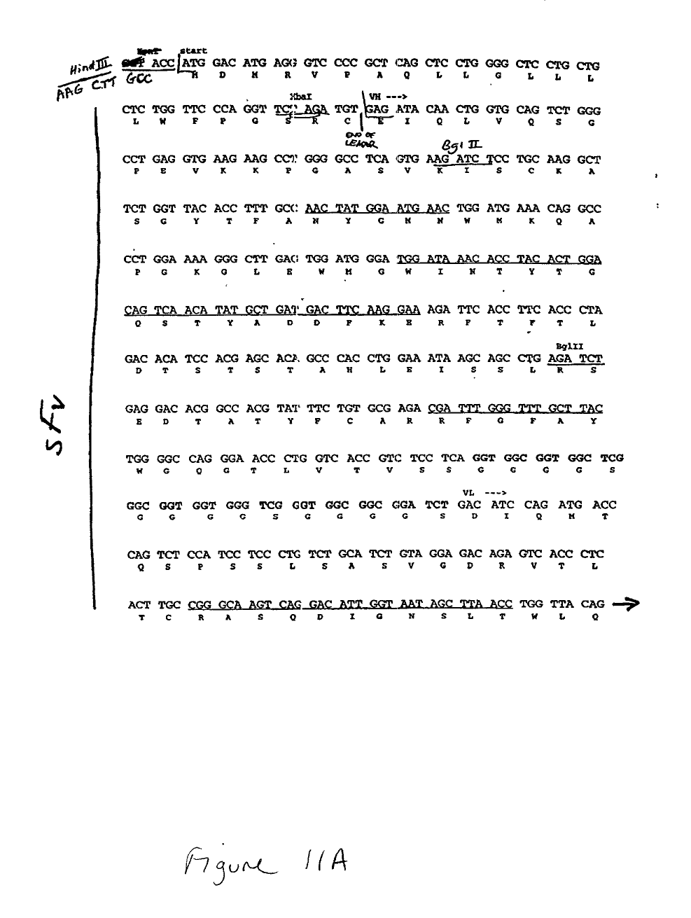

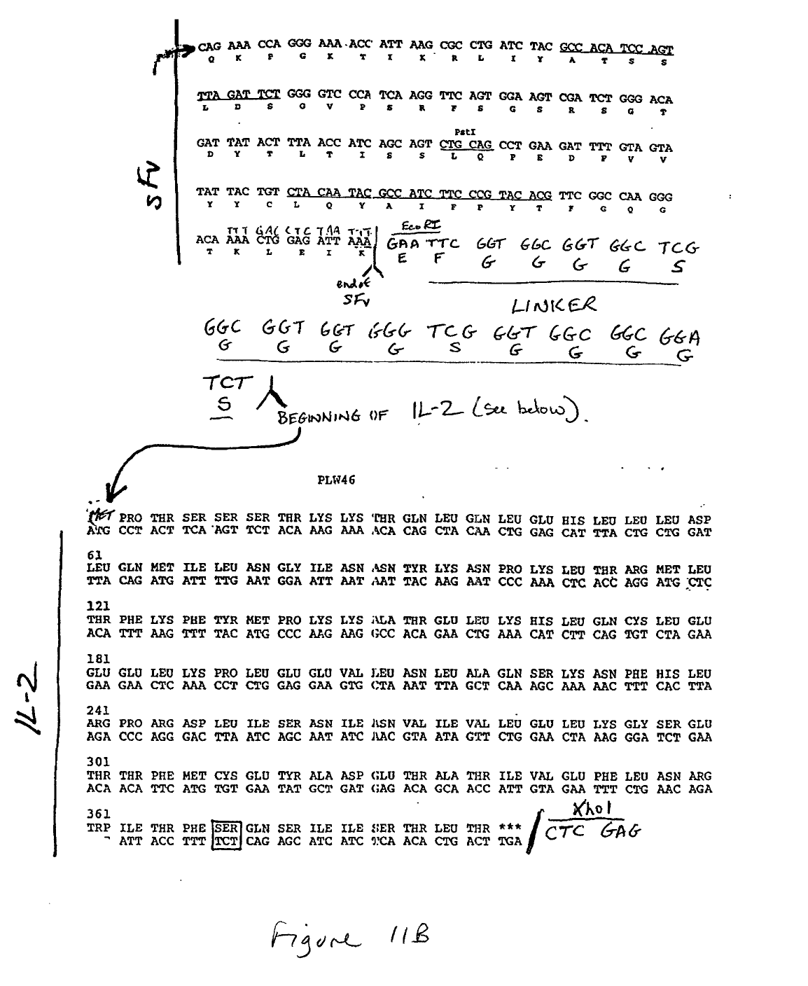

As explained above, the molecules of the present invention are generally produced recombinantly. Thus, polynucleotides encoding the IL-2 polypeptide and p185-specific binding molecule of interest may be expressed and the products subsequently joined together or, preferably, the polynucleotides themselves are joined together to form a chimeric polynucleotide, using techniques well known in the art. A particular example of a recombinant humanized single-chain Fv antibody/IL-2 fusion protein is described below. This recombinant fusion protein consists of humanized variable heavy (V_{H}) and light (V_{L}) domains of monoclonal antibody 520C9 directed against the human HER-2/neu (c-erbB2) proto-oncogene product p185 and human IL-2. The fusion is depicted in FIGS. 11A through 11B (SEQ ID NO:______) and includes a biologically active human IL-2 polypeptide fused to the C-terminus of an anti-p185 antibody by a flexible glycine linker. The fusion retains the immunostimulatory activities of IL-2 and also possesses binding specificity against p185.

These and other molecules for use with the present invention can be made using standard techniques of molecular biology. For example, polynucleotide sequences coding for the above-described molecules can be obtained using recombinant methods, such as by screening cDNA and genomic libraries from cells expressing the gene, or by deriving the gene from a vector known to include the same. Furthermore, the desired gene can be isolated directly from cells and tissues containing the same, using standard techniques, such as phenol extraction and PCR of cDNA or genomic DNA. See, e.g., Sambrook et al., supra, for a description of techniques used to obtain and isolate DNA. The gene of interest can also be produced synthetically, rather than cloned. The molecules can be designed with appropriate codons for the particular sequence. The complete sequence is then assembled from overlapping oligonucleotides prepared by standard methods and assembled into a complete coding sequence. See, e.g., Edge, (1981) Nature 292:756; Nambair et al. (1984) Science 223:1299; and Jay et al. (1984) J. Biol. Chem. 259:6311.

Thus, particular nucleotide sequences can be obtained from vectors harboring the desired sequences or synthesized completely or in part using various oligonucleotide synthesis techniques known in the art, such as site-directed mutagenesis and polymerase chain reaction (PCR) techniques where appropriate. See, e.g., Sambrook, supra. In particular, one method of obtaining nucleotide sequences encoding the desired sequences is by annealing complementary sets of overlapping synthetic oligonucleotides produced in a conventional, automated polynucleotide synthesizer, followed by ligation with an appropriate DNA ligase and amplification of the ligated nucleotide sequence via PVR. See, e.g., Jayaraman et al. (1991) Proc. Natl. Acad. Sci. USA 88:4084-4088. Additionally, oligonucleotide directed synthesis (Jones et al. (1986) Nature 54:75-82), oligonucleotide directed mutagenesis of pre-existing nucleotide regions (Riechmann et al. (1988) Nature 332:323-327 and Verhoeyen et al. (1988) Science 239:1534-1536), and enzymatic filling-in of gapped oligonucleotides using T₄ DNA polymerase (Queen et al. (1989) Proc. Natl. Acad. Sci. USA 86:10029-10033) can be used under the invention to provide molecules having altered or enhanced antigen-binding capabilities, and/or reduced immunogenicity.

Once coding sequences have been prepared or isolated, such sequences can be cloned into any suitable vector or replicon. Numerous cloning vectors are known to those of skill in the art, and the selection of an appropriate cloning vector is a matter of choice. Suitable vectors include, but are not limited to, plasmids, phages, transposons, cosmids, chromosomes or viruses which are capable of replication when associated with the proper control elements.

The coding sequence is then placed under the control of suitable control elements, depending on the system to be used for expression. Thus, the coding sequence can be placed under the control of a promoter, ribosome binding site (for bacterial expression) and, optionally, an operator, so that the DNA sequence of interest is transcribed into RNA by a suitable transformant. The coding sequence may or may not contain a signal peptide or leader sequence which can later be removed by the host in post-translational processing. See, e.g., U.S. Pat. Nos. 4,431,739; 4,425,437; 4,338,397. One system comprises nucleic acid molecules encoding the p185-specific binding molecule and IL-2 polypeptide which are harbored in a single plasmid, either under the control of the same regulatory elements or under the control of separate elements. Similarly, the p185-specific binding molecule and IL-2 polypeptide may be expressed from separate constructs on separate vectors.

In addition to control sequences, it may be desirable to add regulatory sequences which allow for regulation of the expression of the sequences relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector. For example, enhancer elements may be used herein to increase expression levels of the constructs. Examples include the SV40 early gene enhancer (Dijkema et al. (1985) EMBO J. 4:761), the enhancer/promoter derived from the long terminal repeat (LTR) of the Rous Sarcoma Virus (Gorman et al. (1982) Proc. Natl. Acad. Sci. USA 79:6777) and elements derived from human CMV (Boshart et al. (1985) Cell 41:521), such as elements included in the CMV intron A sequence (U.S. Pat. No. 5,688,688). The expression cassette may further include an origin of replication for autonomous replication in a suitable host cell, one or more selectable markers, one or more restriction sites, a potential for high copy number and a strong promoter.

An expression vector is constructed so that the particular coding sequence is located in the vector with the appropriate regulatory sequences, the positioning and orientation of the coding sequence with respect to the control sequences being such that the coding sequence is transcribed under the “control” of the control sequences (i.e., RNA polymerase which binds to the DNA molecule at the control sequences transcribes the coding sequence). Modification of the sequences encoding the molecule of interest may be desirable to achieve this end. For example, in some cases it may be necessary to modify the sequence so that it can be attached to the control sequences in the appropriate orientation; i.e., to maintain the reading frame. The control sequences and other regulatory sequences may be ligated to the coding sequence prior to insertion into a vector. Alternatively, the coding sequence can be cloned directly into an expression vector which already contains the control sequences and an appropriate restriction site.

As explained above, it may also be desirable to produce mutants or analogs of the IL-2 polypeptide. Mutants or analogs may be prepared by the deletion of a portion of the sequence encoding the IL-2 polypeptide, by insertion of a sequence, and/or by substitution of one or more nucleotides within the sequence. Techniques for modifying nucleotide sequences, such as site-directed mutagenesis, and the like, are well known to those skilled in the art. See, e.g., Sambrook et al., supra; Kunkel, T. A. (1985) Proc. Natl. Acad. Sci. USA (1985) 82:448; Geisselsoder et al. (1987) BioTechniques 5:786; Zoller and Smith (1983) Methods Enzymol. 100:468; Dalbie-McFarland et al. (1982) Proc. Natl. Acad. Sci USA 79:6409.

The molecules can be expressed in a wide variety of systems, including insect, mammalian, bacterial, viral and yeast expression systems, all well known in the art. For example, insect cell expression systems, such as baculovirus systems, are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. (“MaxBac” kit). Similarly, bacterial and mammalian cell expression systems are well known in the art and described in, e.g., Sambrook et al., supra. Yeast expression systems are also known in the art and described in, e.g., Yeast Genetic Engineering (Barr et al., eds., 1989) Butterworths, London.

A number of appropriate host cells for use with the above systems are also known. For example, mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human embryonic kidney cells, human hepatocellular carcinoma cells (e.g., Hep G2), Madin-Darby bovine kidney (“MDBK”) cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa califormica, Bombyx mori, Drosophila melanogaster, Spodoptera frugiperda, and Trichoplusia ni.

Nucleic acid molecules comprising nucleotide sequences of interest can be stably integrated into a host cell genome or maintained on a stable episomal element in a suitable host cell using various gene delivery techniques well known in the art. See, e.g., U.S. Pat. No. 5,399,346.

Depending on the expression system and host selected, the molecules are produced by growing host cells transformed by an expression vector described above under conditions whereby the protein is expressed. The expressed protein is then isolated from the host cells and purified. If the expression system secretes the protein into growth media, the product can be purified directly from the media. If it is not secreted, it can be isolated from cell lysates. The selection of the appropriate growth conditions and recovery methods are within the skill of the art.

As explained above, an alternative to recombinant production is chemical conjugation of the separate components. Thus, the IL-2 polypeptide and p185-specific binding molecule may be linked chemically, using standard techniques. For example, the conjugates of the present invention may be made using a variety of bifunctional protein coupling agents known in the art, such as SPDP, IT, bifunctional derivatives of imidoesters such as dimethyl adipimidate Hcl, active esters such as disuccinimidyl suberate, aldehydes such as glutaraldehyde, bisazido compounds such as his(R-azidobenzoyl) hexanediamine, bisdiazonium derivatives such as bis-(R-diazoniumbenzoyl)ethylenediamine, diisocyanates such as tolylene 2,6-diisocyanate, and bis-active fluorine compounds such as 1,5-difluoro-2,4-dinitrobenzene.

Prior to use, the stability and half-life of the fusion molecule may be enhanced by coupling it to a polymer, such as polyethylene glycol (PEG), polypropylene glycol (PPG), and the like. See, e.g., U.S. Pat. Nos. 4,766,106 and 5,206,344.

The IL-2 polypeptide/p185-specific binding molecule (or the humanized anti-p185 antibody) may be formulated into pharmaceutical compositions, described further below, for delivery to a subject. Alternatively, chimeric polynucleotides encoding the fusions (or the humanized anti-p185 antibody) of the present invention may be delivered directly to the subject and expressed in vivo. A number of viral based systems have been developed for direct gene transfer into mammalian cells. In this regard, retroviruses provide a convenient platform for gene delivery systems. A selected nucleotide sequence encoding the desired polypeptide can be inserted into a vector and packaged in retroviral particles using techniques known in the art. The recombinant virus can then be isolated and delivered to a subject. A number of suitable retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109. A number of suitable adenovirus vectors have also been described. Unlike retroviruses which integrate into the host genome, adenoviruses persist extrachromosomally thus minimizing the risks associated with insertional mutagenesis (Haj-Ahmad and Graham (1986) J. Virol. 57:267-274; Bett et al. (1993) J. Virol. 67:5911-5921; Mittereder et al. (1994) Human Gene Therapy 5:717-729; Seth et al. (1994) J. Virol. 68:933-940; Barr et al. (1994) Gene Therapy 1:51-58; Berkner, K. L. (1988) BioTechniques 6:616-629; and Rich et al. (1993) Human Gene Therapy 4:461-476). Various adeno-associated virus (AAV) vector systems have been developed recently for gene delivery. Such systems can include control sequences, such as promoter and polyadenylation sites, as well as selectable markers or reporter genes, enhancer sequences, and other control elements which allow for the induction of transcription. AAV vectors can be readily constructed using techniques well known in the art. See, e.g., U.S. Pat. Nos. 5,173,414 and 5,139,941; International Publication Nos. WO 92/01070 (published Jan. 23, 1992) and WO 93/03769 (published Mar. 4, 1993); Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

Additional viral vectors which will find use for delivering the nucleic acid molecules encoding the molecules of the present invention by gene transfer include those derived from the pox family of viruses, such as vaccinia virus and avian poxvirus. See, e.g., International Publication Nos. WO 91/12882; WO 89/03429; and WO 92/03545.

Molecular conjugate vectors, such as the adenovirus chimeric vectors described in Michael et al. (1993) J. Biol. Chem. 268:6866-6869 and Wagner et al. (1992) Proc. Natl. Acad. Sci. USA 89:6099-6103, can also be used for gene delivery under the invention.

Members of the Alphavirus genus, such as but not limited to vectors derived from the Sindbis and Semliki Forest viruses, will also find use as viral vectors for delivering the gene of interest. For a description of Sinbus-virus derived vectors useful for the practice of the instant methods, see, Dubensky et al., J. Virol. (1996) 70:508-519; and International Publication Nos. WO 95/07995 and WO 96/17072.

The gene of interest can also be delivered without a viral vector. For example, the gene can be packaged in liposomes prior to delivery to the subject or to cells derived therefrom, with or without the accompanying antigen. Lipid encapsulation is generally accomplished using liposomes which are able to stably bind or entrap and retain nucleic acid. For a review of the use of liposomes as carriers for delivery of nucleic acids, see, Hug and Sleight, Biochim. Biophys. Acta. (1991) 1097:1-17; Straubinger et al., in Methods of Enzymology (1983), Vol. 101, pp. 512-527.

Administration and Pharmaceutical Compositions

The invention also provides pharmaceutical compositions comprising the molecules described above, together with one or more pharmaceutically acceptable excipients or vehicles, and optionally other therapeutic and/or prophylactic ingredients. Such excipients include liquids such as water, saline, glycerol, polyethyleneglycol, hyaluronic acid, ethanol, etc. Suitable excipients for nonliquid formulations are also known to those of skill in the art. Pharmaceutically acceptable salts can be used in the compositions of the present invention and include, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients and salts is available in Remington's Pharmaceutical Sciences, 18th Edition (Easton, Pennsylvania: Mack Publishing Company, 1990).

Additionally, auxiliary substances, such as wetting or emulsifying agents, biological buffering substances, surfactants, and the like, may be present in such vehicles. A biological buffer can be virtually any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

For therapeutic purposes, the molecules of the present invention are generally administered directly to the subject. For diagnostic purposes, it may be desirable to administer the humanized anti-p185 antibody in vitro, e.g., to biological samples derived from the subject, such as cells, urine, blood, saliva, etc. Alternatively, diagnosis may also be carried out in vivo using the humanized anti-p185 antibody.

Thus, once formulated, the compositions of the invention are generally administered parenterally. Administration can include, for example, administration intravenously, intra-arterially, intra-articularly (e.g., into the knee), subcutaneously, intradermally, intramuscularly, transdermally, intranasally, mucosally, and by aerosol administration. For example, the composition can be administered by inhalation, e.g., as a nasal or mouth spray or aerosol. The compositions may also be delivered in situ, e.g., by implantation.

A pharmaceutically or therapeutically effective amount of the composition will be delivered to the subject. The precise effective amount will vary from subject to subject and will depend upon the species, age, the subject's size and health, the nature and extent of the condition being treated, recommendations of the treating physician, and the therapeutics or combination of therapeutics selected for administration. Thus, the effective amount for a given situation can be determined by routine experimentation. For purposes of the present invention, generally a therapeutic amount will be in the range of about 0.1 μg/kg to about 100 mg/kg, more preferably about 1 μg/kg to about 1 mg/kg, and most preferably about 2 μg/kg to about 500 μg/kg, in at least one dose. The subject may be administered as many doses as is required to reduce and/or alleviate the signs, symptoms, or causes of the disorder in question, or bring about any other desired alteration of a biological system.

Pharmacology and Utility

In a preferred embodiment, the fusions of this invention are useful for treating disease indications in vivo, particularly those diseases characterized by overexpression of the p185 protein. In more preferred embodiments, the cells are cancer cells, such as malignant and/or benign tumors which express the p185 protein, including, without limitation, breast, renal, gastric, gastrointestinal and salivary gland tumors, and a variety of other human adenocarcinomas.

In an alternative embodiment, the humanized anti-p185 antibody of the invention is useful for detecting or monitoring the presence of a disease in a mammal suspected of having said disease. In this embodiment, a biological sample is obtained from a subject suspected of having cancer. The sample is then reacted with the humanized anti-p185 antibody, which includes a detectable label, such as a radiolabel, fluorochrome, or an enzyme. Presence of cells expressing the p185 antigen is indicative of cancer. The method of detection can take any of many forms, including standard assays such as RIA, ELISA, precipitation, agglutination, complement fixation and immunofluorescene. Assays for the ability of molecules to bind p185 are described in, e.g., U.S. Pat. Nos. 5,677,171; 5,720,937; 5,720,934; 5,725,856; 5,770,195; 5,772,997.

Deposits of Strains Useful in Practicing the Invention

A deposit of biologically pure cultures of the following strains was made with the American Type Culture Collection (ATCC), 10801 University Boulevard, Manassas, Va. The accession number indicated was assigned after successful viability testing, and the requisite fees were paid. The deposits were made under the provisions of the Budapest Treaty on the International Recognition of the Deposit of Microorganisms for the Purpose of Patent Procedure and the Regulations thereunder (Budapest Treaty). This assures maintenance of viable cultures for a period of thirty (30) years from the date of deposit and at least five (5) years after the most recent request for the furnishing of a sample of the deposit by the depository. The organisms will be made available by the ATCC under the terms of the Budapest Treaty, which assures permanent and unrestricted availability of the cultures to one determined by the U.S. Commissioner of Patents and Trademarks to be entitled thereto according to 35 USC §122 and the Commissioner's rules pursuant thereto (including 37 CFR §1.12). Upon the granting of a patent, all restrictions on the availability to the public of the deposited cultures will be irrevocably removed.

These deposits are provided merely as convenience to those of skill in the art, and are not an admission that a deposit is required under 35 USC §112. The nucleic acid sequences of these hybridomas, as well as the amino acid sequences of the polypeptides encoded thereby, are incorporated herein by reference and are controlling in the event of any conflict with the description herein. A license may be required to make, use, or sell the deposited materials, and no such license is hereby granted.

| Strain | Deposit Date | ATCC No. |
|---|---|---|
| Hybridoma secreting | | |
| 520C9 | January 8, 1985 | HB8696 |
| pLW46 (in E. coli) | September 26, 1983 | 39,452 |
| pLW1 (in E. coli) | August 4, 1983 | 39,405 |

III. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

Restriction and modifying enzymes, as well as PCR reagents were purchased from commercial sources, and used according to the manufacturers' directions. In the cloning of DNA fragments, except where noted, all DNA manipulations were done according to standard procedures. See, e.g., Sambrook et al., supra. Restriction enzymes, T₄ DNA ligase, E. coli, DNA polymerase I, Klenow fragment, and other biological reagents were purchased from commercial suppliers and used according to the manufacturers' directions. Double-stranded DNA fragments were separated on agarose gels.

Data are presented as means±standard deviation (S.D.). Statistical differences between the groups were determined by analysis of variance. If a significant difference between these groups was demonstrated, an unpaired Student's t-test was performed for each point. For all analyses, p values <0.05 were considered statistically significant.

### EXAMPLE 1

### Construction of Plasmids pLW46 and pLW42 Encoding IL-2 Polypeptides

Plasmids pLW46 and pLW42, encoding an active IL-2 polypeptide and an inactive IL-2 protein, respectively, were produced as described in U.S. Pat. No. 4,518,584, incorporated herein by reference in its entirety. The nuclcotide sequence for a cDNA clone coding for human IL-2, procedures for preparing IL-2 cDNA libraries, and screening same for IL-2 are described by Taniguchi et al. Nature (1983) 24:305.

Briefly, the IL-2 gene was obtained from plasmid pLW1 (ATCC Accession No. 39,405) which contains the IL-2 gene under the control of the E. coil trp promoter and cloned into an M13 phage vector (J. Messing, “Third Cleveland Symposium on Macromolecules: Recombinant DNA,” A Walton, Ed., Elsevier Press, 1981, pp143-153). The construct was transformed into competent cells of E.coli strain JM 103 and plated on Xgal indicator plates (Messing et al. Nucleic Acids Res (1981) 9:309-321). Plaques containing recombinant phage (white plaques) were picked, inoculated into a fresh culture of JM 103 and minipreps of replicative form (RF) molecules prepared from the infected cells (Birnboim H. D. and Doly J. Nucleic Acid Res (1979) 7:1513-1523). The RF molecules were digested with various restriction enzymes to identify the clones containing the IL-2 insert, designated M13-IL2 clones herein.

Single-stranded phage DNA was prepared from M13-IL2 clones to serve as a template for oligonucleotide-directed mutagenesis, as described in U.S. Pat. No. 4,518,584. IL-2 contains cysteine residues at amino acid positions 58, 105 and 125 (see, FIG. 8). Based on the nucleotide sequences of the portions of the IL-2 gene that contain the codons for cysteine residues 58 and 125, oligonucleotide primers were designed and synthesized for mutating the codons for these residues to codons for ser. These oligonucleotides have the following sequences:

CTTCTAGAGACTGCAGATGTTTC (SEQ ID NO:______) “DM27” to change cys 58 to ser,

GATGATGCTCTGAGAAAAGGTAATC (SEQ ID NO:______) “DM29” to change cys 125 to ser.

Forty picomoles of each oligonucleotide were kinased separately in the presence of 0.1 mM ATP, 50 mM Tris-HCl, pH 8.0, 10 mM MgCl.sub.2, 5 mM DTT and 9 units of T4 kinase in 50 μl at 37° C. for 1 hr. Each of the kinased primers (10 pmoles) was hybridized to 2.6 μg of ss M13-IL2 DNA in 15 μl of a mixture containing 100 mM NaCl, 20 mM Tris-HCl, pH 7.9, 20 mM MgCl₂ and 20 mM β-mercaptoethanol, by heating at 67° C. for 5 min. and 42° C. for 25 min. The annealed mixtures were chilled on ice and then adjusted to a final volume of 25 μl of a reaction mixture containing 0.5 mM of each dNTP, 17 mM Tris-HCl, pH 7.9, 17 mM MgCl₂, 83 mM NaCl, 17 mM β-mercaptoethanol, 5 units of DNA polymerase I Klenow fragment, 0.5 mM ATP and 2 units of T4 DNA ligase, incubated at 37° C. for 5 hr. The reactions were terminated by heating to 80° C. and the reaction mixtures used to transform competent JM103 cells, plated onto agar plates and incubated overnight to obtain phage plaques.

Plates containing mutagenized M13-IL2 plaques as well as plates containing unmutagenized M13-IL2 phage plaques, were chilled to 4° C. and phage plaques from each plate were transferred onto two nitrocellulose filter circles by layering a dry filter on the agar plate for 5 min. for the first filter and 15 min. for the second filter. The filters were then placed on thick filter papers soaked in 0.2N NaOH, 1.5M NaCl and 0.2% Triton for 5 min., and neutralized by layering onto filter papers soaked with 0.5M Tris-HCl, pH 7.5, and 1.5M NaCl for another 5 min. The filters were washed in a similar fashion twice on filters soaked in 2× SSC, dried and then baked in a vacuum oven at 80° C. for 2 hr. The duplicate filters were pre-hybridized at 42° C. for 4 hr. with 10 ml per filter of DNA hybridization buffer (5× SSC, pH 7.0, 4× Denhardts solution (polyvinylpyrrolidine, ficoll and bovine serum albumin, 1×=0.02% of each), 0.1% SDS, 50 mM sodium phosphate buffer, pH 7.0 and 100 μg/ml of denatured salmon sperm DNA. ³²P-labelled probes were prepared by kinasing the oligonucleotide primers with labelled ATP. The filters were hybridized to 0.1×10⁵ cpm/ml of ³²P-labelled primers in 5 ml per filter of DNA hybridization buffer at 42° C. for 8 hr. The filters were washed twice at 50° C. for 30 min. each in washing buffers containing 0.1% SDS and 2× SSC, and twice at 50° C. for 30 min. each with 0.1% SDS and 0.2× SSC. The filters were air dried and autoradiographed at −70° C. for 2-3 days.

Oligonucleotide primer DM29 was designed to create a new DdeI restriction site in the mutagenized clone. Therefore, RF-DNA from a number of the clones which hybridized with the kinased primer were digested with the restriction enzyme DdeI. One of the mutagenized M13-IL2 placques which hybridized with the primer DM29 and had a new DdeI restriction site (M13-LW46) was picked and inoculated into a culture of JM103, ssDNA was prepared from the culture supernatant and dsRF-DNA was prepared from the cell pellet.

The oligonucleotide primer DM27 was designed to create a new PstI restriction site instead of a DdeI site. Therefore, the plaques that hybridized to this primer were screened for the presence of a new PstI site. One such phage plaque was identified (M13-LW42) and ssDNA and RF-DNA prepared from it. The DNA from these clones were sequenced to confirm that the target TGT codons for cysteine had been converted to a TCT codon for serine.

RF-DNA from M13-LW42 and M13-LW46 were each digested with restriction enzymes HindIII and BanII and the insert fragments purified from a 1% agarose gel. Similarly, the plasmid pTrp3, including the E. coli trp promoter, was digested with HindIII and BanII, the large plasmid fragment containing the trp promoter was purified on an agarose gel and then ligated with each of the insert fragments isolated from M13-LW42 and M13-LW46. The ligated plasmids were transformed into competent E. coli K12 strain MM294. The plasmid DNAs from these transformants were analyzed by restriction enzyme mapping to confirm the presence of the plasmids pLW42 and pLW46. When each of these individual clones were grown in the absence of tryptophane to induce the trp promoter and cell free extracts analyzed on SDS-polyacrylamide gels, both clones, pLW42 and pLW46, were shown to synthesize a 14.5 kd protein similar to that found in the positive control, pLW21, which has been demonstrated to synthesize a 14.4 kd IL-2 protein. When these same extracts were subjected to assay for IL-2 activity on mouse HT-2 cells (Watson J. J Exp Med (1979) 150:1570-1519), only clones pLW21 (positive control) and pLW46 had significant amounts of IL-2 activity (Table 1 below), indicating that cys-58 is necessary for biological activity and changing it to a serine (pLW42) resulted in the loss of biological activity. Cys-125, on the other hand must not be necessary for biological activity because changing it to ser-125 (pLW46) did not affect the biological activity.

TABLE 1

| Clone | IL-2 Activity (μ/ml) |
|---|---|
| pIL2-7 (negative control) | 1 |
| pLW21 (positive control) | 113,000 |
| pLW42 | 660 |
| pLW46 | 123,000 |

As compared to the coding strand of the native human IL-2 gene, clone pLW46 has a single base change of G to C at nucleotide position 374 which causes a change of cys to ser at amino acid 125. The clone also has an initial N-terminal met (which is processed off), and lacks the initial N-terminal ala of the native molecule. This mutein is designated des-alanyl(ala) IL-2ₛₑᵣ₁₂₅. A sample of E. coli K12 strain MM294 transformed with pLW46 was deposited in the ATCC on Sep. 26, 1983 and has been assigned ATCC Accession Number 39,452.

### EXAMPLE 2

### Construction of Plasmid pAcHCs-520C9sFv Encoding a Humanized Anti-p185 Antibody

Murine monoclonal antibody 520C9, an anti-p185 antibody, is described in, e.g., U.S. Pat. Nos. 4,753,894, 5,169,774, 5,629,197 and the hybridoma secreting the antibody has been deposited at the ATCC with Accession No. HB8696. Baculovirus expression plasmid pAcHCs-520C9sFv, containing a humanized 816 bp sFv cDNA fragment (FIG. 9) of 520C9, a p185 antibody, was constructed as follows. The murine heavy and light chain sequences were compared to a database of human antibody sequences, and those human sequences with frameworks which most closely matched were selected. Taking into consideration positions which determine the canonical confirmation of the variable regions, and positions which have been identified as possibly contacting antigen in published reports discussing other antibodies, human sequences were selected which most closely matched each heavy and light chain as a blueprint for humanization.

Each residue of the variable regions was identified as exposed, mostly exposed, partially buried, or buried based on data from solved crystal structures of variable regions. Positions with residues described as exposed or mostly exposed were switched to the corresponding human amino acid, while at those positions described as buried or partially buried, the murine amino acids were maintained. The humanized variable regions were constructed by a technique using overlapping oligonucleotides with 5′ phosphates which were allowed to anneal, were ligated, and then were used as target for PCR amplification. Oligos ranging between 27 and 66 nucleotides were employed. These oligos formed either 14 complementary pairs in the construction of the 520C9 light chain, or 16 complementary pairs in the construction of the 520C9 heavy chain. When the oligo pairs were annealed, they produced fragments with sticky ends of between 8 and 14 nucleotides. Oligos were chosen so that the Tms of any secondary stern-loop structures that could be formed were significantly less than the Tms for the complementary pairs, so that there were no large similarities amongst the sticky ends created, and so that end nucleotides of the annealed pairs were either G or C. For the construction of each variable region, 5 μl of each oligo at a concentration of 100 picomoles/μl were pooled, and then 5μl of this mix was diluted to 50 μl with 10×T4 DNA Ligase buffer containing ATP at a final concentration of 1 mM and water. The mix was subjected to the following thermal manipulations: 1) Heat to 95° C. over 30 min. and then hold at 95° C. for 5 min. 2) Ramp to 75° C. over 20 min. and then hold at 75° C. for 10 min. to allow for the complementary pairs to anneal at a temperature which discourages the formation of unwanted secondary structures, 3) Ramp to 37° C. over 20 min. and then hold at 37° C. for 10 min. to allow for the sticky ends generated in step 2 to anneal, 4) Ramp to 12° C. over 35 min. 1 μl of T4 DNA Ligase (6 Weiss units) was then added and the mixtures were allowed to ligate overnight at 12° C.

Each ligated variable region was then amplified by PCR, with the 5′-most sense oligo and the 3′-most antisense oligo serving as primers. 1 ul of each ligation reaction was combined in a 100 μl PCR with 1× PCR buffer, 20 μM dNTPs, 20 picomoles each primer, and 2.5 units Taq DNA Polymerase. Cycling was as follows: 1) Denature by heating to 95° C. over 30 min. and then holding at 95° C. for one min., 2) Anneal by cooling to 65° C. over 30 min. and then holding at 65° C. for one min., 3) Extend by heating to 72° C. over 30 min. and then holding at 72° C. for two min. This was repeated over 15 cycles and then followed by a 10 min. incubation at 72° C. The PCRs were extracted with chloroform and DNA fragments of the expected sizes were purified from agarose gels after electrophoresis.

520C9 humanized Fabs and humanized single-chain antibodies (huscs) were made as follows. All constructs were expressed from recombinant baculovirus in Sf9 insect cells. Vector pHCB201 was used to express the VH+CH1 sequences of 520C9 Fabs, while vector pLCB201 was used to express the VL+CL sequences of 520C9 Fabs.

All huscs were expressed from a modified pHCB201 vector. Plasmid pHCB201 and pLCB201 are derived from the baculovirus expression vector pAcC13, which directs expression from a baculovirus polyhedrin promoter. In pHCB201, inserted between the KpnI and SmaI sites of the pAcC13 MCS, is a 57 base-pair KpnI-XbaI fragment containing a start codon and codons −21 through −4 of the human kappa chain signal sequence, an XbaI-SpeI fragment containing a 20 base pair non-coding spacer, an SpeI-BamHI fragment containing 234 base pairs coding for residues 135 through 222 of human IgG1 CH1, and a 21 base pair BamHI-SmaI fragment containing the six codons of the glu/glu epitope for antibody E5 (the peptide sequence of this tag is EYMPME, from Chiron Mimotopes) and a TGA stop codon. Plasmid pLCB201 is identical except that the SpeI-BamHI fragment is 244 base pairs coding for the last nucleotide of codon 133 and codons 134 through 214 of human kappa CL. In each vector, the XbaI site codes for residues −3 and −2 of the signal peptide (TCT=S and AGA=R) while in pHCB201 SpeI codes for residues 133 and 134 of human IgGI CH1 (ACT=T and AGT=S) and in pLCB201 within the SpeI site is the codon for position 132 of human kappa CL (CTA=L) and the first two nucleotides of the codon for human kappa CL position 133 (GTG-V). Heavy chain variable regions were cloned into pHCB201 on XbaI-SpeI fragments coding for residues −3 to 134, and light chain variable regions were cloned into pLCB201 on XbaI-SpeI fragments coding for residues −3 through 133. The XbaI site and residue −1 were incorporated in the construction of the variable regions by the overlapping oligo protocol; however, residues 114 through 134 of CH1 and 108 through 133 of CL had to be added through a two step PCR method. In the first step of each, two PCRs produced fragments with complementary sequences spanning the end of the variable regions and the beginning of the constant regions. One fragment was made with a sense primer annealing to the 5′ end of the variable region construct which included the XbaI cloning site, and an antisense primer annealing to the 3′ end of the variable region which included a long tail with sequence matching the 5′ end of the constant region. The second fragment was made with a sense primer annealing to the 5′ end of the constant region which included a long tail with sequence matching the 3′ end of the variable region, and an antisense primer annealing at positions 132-134 of each constant region and including the SpeI cloning site. Two fragments were thus produced with an area of complementary sequence surrounding the variable-constant border. These fragments were isolated and used together in a second PCR with the XbaI containing sense primer which produced the first fragment and the SpeI containing antisense primer which produced the second fragment.

The first step PCRs were in 100 μl with approximately 200 ng of target DNA, 1× PCR buffer, 20 μM dNTPs, 20 picomoles each primer, and 2.5 units Taq DNA Polymerase. Cycling was as follows: 1) Denature by heating to 95° C. over 30 min. and then holding at 95° C. for one min., 2) Anneal by cooling to 50° C. over 30 min. and then holding at 50° C. for one min., 3) Extend by heating to 72° C. over 30 min. and then holding at 72° C. for two min. This was repeated over 20 cycles and then followed by a 10 min. incubation at 72° C. Target DNA was either the variable region product of the overlapping oligo protocol, or plasmid containing previously cloned human IgG1 heavy chain or human kappa light chain. The PCRs were chloroform-extracted and purified, and then combined at approximately 1 ng each in 100 μl second step PCRs with 1× PCR buffer, 20 μM dNTPs, 100 picomoles each primer, and 2.5 units Taq DNA Polymerase. Cycling was as follows: 1) Denature by heating to 95° C. over 30 min. and then holding at 95° C. for one min., 2) Anneal by cooling to 50° C. over 30 min. and then holding at 50° C. for two min., 3) Extend by heating to 72° C. over 30 min. and then holding at 72° C. for three min. This was repeated over 15 cycles and then followed by a 10 min. incubation at 72° C. After cycling, the PCRs were subjected to agarose, gel electrophoresis and fragments of the size expected for the fusion products were purified from the gel, digested with XbaI and SpeI, and ligated into pHCB201 or pLCB201. Clones were sequenced to identify those with the desired sequences. The 520C9 Fab-expressing plasmids are named pAcHC-520C9H and pAcLC-520C9L.

The 520C9 husc was constructed in the heavy chain-light chain orientation, with a central peptide linker composed of three repeats of a GGGGS motif. A two step overlapping PCR method similar to that used in the fusion of the variable regions to the constant regions of the Fab molecules was employed. In the first step reactions, the heavy chain PCRs used sense primers containing XbaI sites that were identical to those used in the Fab constructions and antisense primers which annealed to the 3′ end the heavy chain variable region and which incorporated a tail with the 5′ section of the central linker coding sequence. The light chain PCRs used sense primers which annealed to the 5′ end of the light chain variable region and incorporated a tail with the 3′ end of the central linker coding sequence which overlapped the tail created on the heavy chain. The antisense primer for the light chain PCR annealed to the 3′ end of the variable region and incorporated a BamHI cloning site immediately following it. PCR conditions were identical to those described for the variable-constant fusions. The products of the second step PCRs were fragments with the two variable regions connected by the central linker coding sequence on XbaI-BamHI fragments. These fragments were purified from agarose gels after electrophoresis, restriction digested with XbaI and BamHI, ligated into XbaI-BamHI digested pHCB201 vector, and sequenced to identify those with the desired sequences. Ligating into pHCB201 digested with XbaI and BamHI places the insert between the human kappa signal sequence and the glu/glu epitope, and thus removes all constant region codons. This plasmid was named pAcHCs-520C9sFv and expressed the 520C9 husc depicted in FIG. 9.

The husc produced by pAcHCs-520C9sFv was expressed from recombinant baculovirus in Sf9 insect cells. The DNA to be expressed is under the control of the baculovirus polyhedrin late promoter. This recombines within the baculovirus genome, producing a population of recombinant virus used to infect Sf9 cells (recombinant virus protocol). A 10 cm culture dish was seeded with 3.5 million Sf9 cells in 10 ml medium containing 10% FCS, and incubated 24 hrs at 27° C. The medium was discarded and 1 ml of virus-bearing supernatant was added along with 3 ml of ISFM-7 medium containing 5% FCS and 0.1 ml of Erythrogen. The infected cells were incubated 5 days at 27° C. in a moist container. The medium was then collected, spun 10 min. at 14,000 rpm at 4° C., and the supernatant stored at 4° C. Western blots were done to assay culture supernatants for the presence of a glu/glu tagged protein of the expected molecular weight. 20 μl of supernatant was run on precast 4-20% SDS PAGE gels (Novex) using either nonreducing or reducing sample buffer, and transferred to nitrocellulose membrane in a Novex transfer chamber (25 volts for 40 min.). The membrane was blocked for 1 hr. at room temp. in PBS/0.1% Tween 20 containing 5% normal mouse serum, washed 15 min. and 2×5 min. with PBS/T, and incubated 1 hr./room temp. in PBS/T containing 0.5 μg/ml E5-HRP probe and 3% FCS. The membrane was then washed 15 min. and 4×5 min. with PBS/T, incubated with ECL reagents (Amersham) and exposed to film, confirming the presence of the 520C9 husc.

### EXAMPLE 3

### Construction of Plasmids pcDNA-H520C9sFv-hIL-2 and pcDNA-H520C9sFv-mhIL-2 Encoding Humanized Anti-p185 Antibody-IL2 Fusions

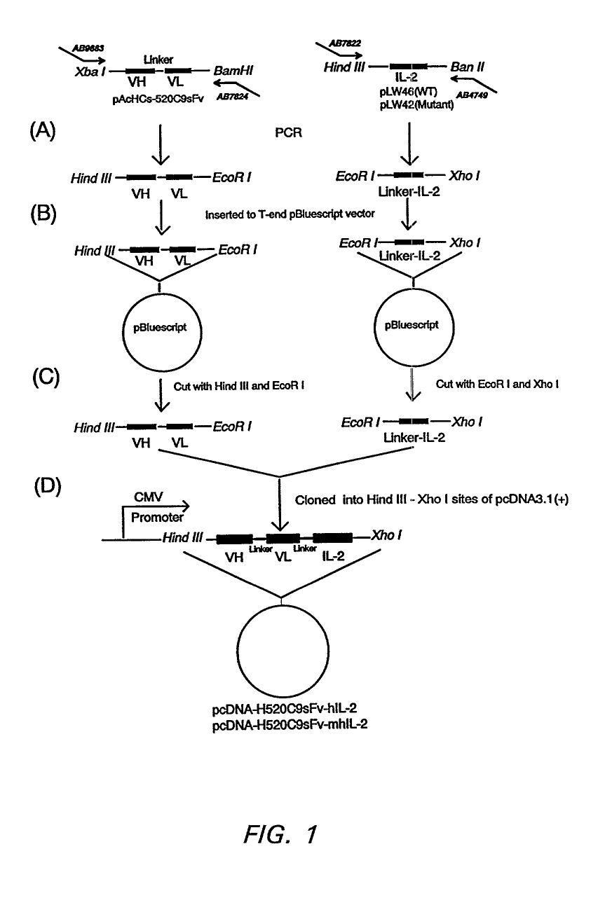

Mammalian expression plasmids encoding the humanized sFv antibody/IL-2 fusion proteins were constructed as described in FIG. 1 using baculovirus expression plasmid pAcHCs-520C9sFv, described above, containing a humanized 816-bp 520C9 sFv cDNA fragment, and the plasmids pLW46 and plasmid pLW42, also described above, containing either active or inactive human IL-2 cDNA fragments, respectively. Primers used for the amplification of 520C9 sFv and human IL-2 cDNA fragments from the above plasmids were synthesized at the Institute for Molecular Biology (MOBIX), McMaster University (Hamilton, ON). The forward primer AB9883 (5′-CTT AAG CTT GCC ACC ATG GAC ATG AGG GTC CCC GCT-3′) (SEQ ID NO:______) used to amplify the sFv cDNA contained a Kozak consensus sequence prior to the initiating ATG and a terminal HindIII restriction site. The reverse primer AB7824 (5′-CC GAA TTC TTT AAT CTC CAG TTT TGT CCC TTG GGC-3′) (SEQ ID NO:______) contained an EcoRI site 3′ to the sFv cDNA fragment. The inactive human IL-2 and active human IL-2 cDNA fragments derived from pLW42 and pLW46 were subjected to PCR amplification using the forward primer AB7822 (5′-GGG GAA TTC GGT GGC GGT GGC TCG GGC GGT GGT GGG TCG GGT GGC GGC GGA TCT ATG CCT ACT TCA AGT TCT ACA AAG AAA ACA CAG-3′) (SEQ ID NO:______), which contained a terminal EcoRI site and a coding sequence for a 15 amino acid, glycine-rich linker peptide (NH₂-GGGGSGGGGSGGGGS-COOH) (SEQ ID NO:______) 5′ to the human IL-2 cDNA fragment. The reverse primer AB4749 (5′-GAC CTC GAG TCA GTG TTG AGA TGA T-3′) (SEQ ID NO:______) contained a terminal XhoI site adjacent to the termination codon of the human IL-2 cDNA fragment. PCR products were purified from 0.8% agarose gels and ligated into T-ended pBluescript using T4 DNA ligase (Gibco/BRL, Burlington, ON). The ligation mixture was then used to transform competent DH5α cells (Gibco/BRL). Plasmids with inserts were digested with HindIII and EcoRI for the sFv, or EcoRI and XhoI for the human IL-2 cDNA fragments. These cDNA fragments were purified from agarose gels using QIAGEN Gel Purification Kit (Qiagen, Chatsworth, Calif.) and ligated into the HindIII/XhoI site of the mammalian expression vector pcDNA3.1(+) (Invitrogen, San Diego, Calif.) to produce the recombinant plasmids pcDNA-H520C9sFv-hIL-2 and pcDNA-H520C9sFv-mhIL-2. Fidelity of the PCR-amplified constructs was confirmed by fluorescence-based double-stranded DNA sequencing (MOBIX). The ligation mixture was then used to transform competent DH5α cells. Transformants containing both the humanized 520C9 sFv and IL-2 cDNA fragments were confirmed by restriction enzyme analysis. The constructs were subsequently purified using QIAGEN Plasmid Midi Kits and dissolved in Tris-EDTA buffer (pH 7.4) to a concentration of 0.4 mg/ml.

### EXAMPLE 4

### Expression of cDNAs Encoding H520C9sFv-hIL-2 and H520C9sFv-mhIL-2

Baby hamster kidney (BHK) cells used for stable expression of the cDNAs encoding H520C9sFv-hIL-2 or H520C9sFv-mhIL-2 were maintained in Dulbecco's modified Eagle's medium (DMEM) (Gibco/BRL) containing 10% fetal calf serum (FCS; HyClone Laboratories, Logan, Utah) at 37° C. in a 5% CO₂ incubator. Cells grown to 20% confluency were transfected with 5 μg of the plasmid constructs using 30 μl of SuperFect Transfection Reagent (Qiagen) as described by the manufacturer. As a negative control, pcDNA3.1(+) was used to transfect BHK cells under the same conditions. Stable transfectants were selected in DMEM containing 10% FCS and 600 μg/ml G418 (Gibco/BRL) for two weeks, after which the concentration of G418 was increased to 1.2 mg/ml. Clones secreting the H520C9sFv-hIL-2 fusion protein were identified by ELISA using either a mouse anti-human IL-2 mAb MAB202 (R&D Systems, Minneapolis, MN) or a rabbit anti-human IL-2 polyclonal antibody EP100 (Genzyme, Cambridge, Mass.).

In brief, 100 μl of supernatant from different clones was added to a 96-well plate coated with MAB202 (1:250 dilution), and incubated at 37° C. for 2 hr. After three washes with PBST, the plate was incubated with EP100 (1:200 dilution) at 37° C. for 1 hr., washed and then incubated with horseradish peroxidase (HRP)-conjugated goat anti-rabbit IgG polyclonal antibody (Gibco/BRL) (1:1000 dilution) for 1 hr. at 37° C. After three additional washes with PBST, color development was performed with peroxidase substrate (Bio-Rad, Hercules, Calif.) and the resulting OD determined at 405 nM in a microplate reader (Bio-Tek Instrument Inc., Winooski, Vt.). Clones producing the highest amount of fusion protein were selected and grown in DMEM containing 10% FCS for 72 hr. The conditioned medium was collected for subsequent precipitation and analysis.

H520C9sFv-hIL-2 fusion protein was immunoprecipitated using MAB202 coupled with CNBr-activated Sepharose CL-4B (Pharmacia Biotech, Baie d'Urfé, QC). 25 μl of a 1:1 (v/v) slurry of the absorbent suspended in 20 mM Tris-HCl buffer (pH 7.8) was added to 1.5 ml of the conditioned medium with gentle shaking at 4° C. for 4 hr. The Sepharose beads were washed three times using Tris-HCl buffer, then 50 μl of 1× SDS-PAGE sample buffer containing β-mercaptoethanol was added to the beads. After heating for 3 min. at 100° C., the beads were pelleted by centrifugation and the supernatant collected for immunoblot analysis which was performed as follows. 20 μl of 50-fold concentrated conditioned media or 20 μl of the immunoprecipitate were heated to 100° C. for 3 min. and then separated on a 15% SDS-polyacrylamide gel under reducing conditions. Proteins were electroblotted onto nitrocellulose membranes (Bio-Rad, Richmond, Calif.) and the efficiency of protein transfer was determined by staining the blots with Ponceau S (Sigma, St. Louis, Mo.). Membranes were washed briefly in TBST to remove the Ponceau S stain, blocked in TBST containing 5% skimmed milk at 4° C. overnight and incubated at room temperature for 2 hr. with EP100 (1:1000 dilution). Following three 20 min. washes in TBST, the membranes were incubated for 2 hr. with a HRP-conjugated goat anti-rabbit IgG polyclonal antibody (1:2000 dilution). After washing in TBST, membranes were developed with the Renaissance Chemiluminescence Reagent Kit (DuPont, Boston, Mass.) as described by the manufacturer.

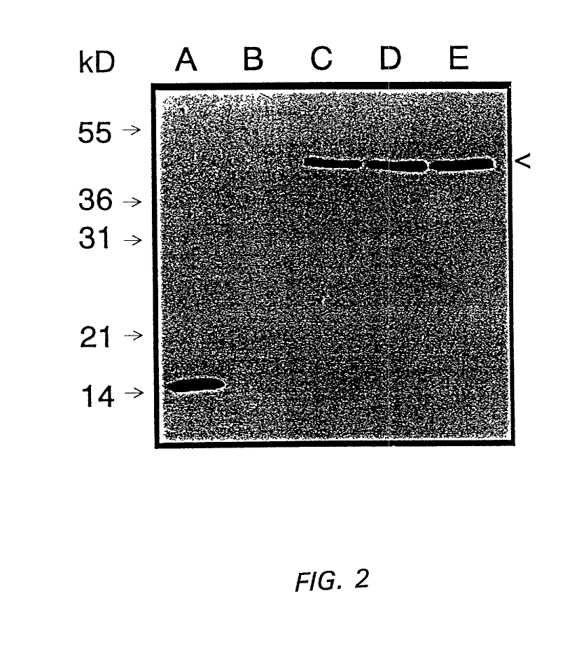

As shown in FIG. 2, a single band of 45 kD was observed in the conditioned media from BHK cells transfected with either pcDNA-H520C9sFv-hIL-2 (lane C), pcDNA-H520C9sFv-mhIL-2 (lane D), or the MAB202 immunoprecipitate (lane E), but not in the conditioned medium from BHK transfected with pcDNA3.1(+) (lane B). Furthermore, the migration positions of these fusion proteins on SDS-PAGE are consistent with their predicted molecular mass. As a positive control, EP100 also recognized rhIL-2 (lane A).

For affinity purification of the fusion protein, the conditioned medium was applied to a MAB202-Sepharose CL-4B affinity column at a flow rate of 5 column vol/h. The column was then washed three times with 20 mM Tris-HCl buffer (pH 7.8) and the fusion protein was eluted with Pierce Gentle Ag/Ab Elution Buffer (Rockford, Ill.) as described by the manufacturer.

The concentration of IL-2, as a constitutive component of H520C9sFv-hIL-2 in the conditioned medium was approximately 0.467 nM (data not shown) as determined by quantitative indirect ELISA.

The nucleotide sequence (SEQ ID NO:______) and corresponding amino acid sequence (SEQ ID NO:______) of H520C9sFv-hIL-2 are shown in FIGS. 11A through 11B.

### EXAMPLE 5

### IL-2 Activity of H520C9sFv-hIL-2 and H520C9sFv-mhIL-2

Biological activity of the fusion proteins was determined by standard IL-2-dependent CTLL-2 cell proliferation (Gillis et al. J Immunol (1978) 120:2027-2030) and cytotoxicity assays (Carter et al. Proc Natl Acad Sci USA (1992) 89: 4285-4289). CTLL-2 cells deprived of rhIL-2 (Chiron, Emeryville, Calif.) for 3 days were seeded into a 96-well plate at 1×10⁴ cells per well and incubated with either serially diluted rhIL-2 standard or fusion protein at 37° C. in a 5% CO₂ incubator. After 24 hr., 1 μCi of [³H]-thymidine (DuPont, Boston, Mass.) was added to each well and the cells incubated for an additional 12 hr. Cells were harvested onto glass fibre filters (Whittaker Corp., Walkersville, Md.), dried and the incorporated radioactivity counted in a Beckman scintillation counter (Fullerton, Calif.).

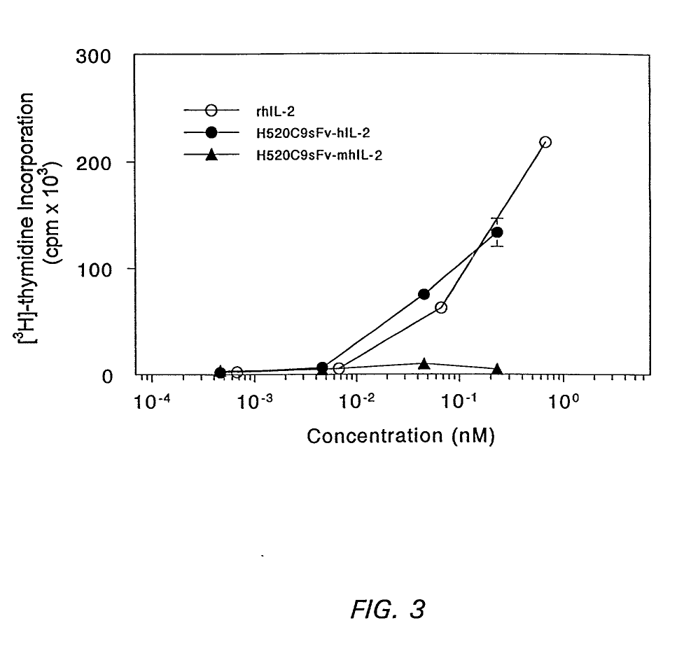

The activity of the H520C9sFv-hIL-2 fusion protein to support the proliferation of IL-2-dependent CTLL-2 cells and to generate LAK cells was compared to those of rhIL-2. As shown in FIG. 3, conditioned media from BHK cells transfected with pcDNA-H520C9sFv-hIL-2, but not pcDNA-H520C9sFv-mhIL2, possessed similar IL-2 bioactivity to rhIL-2 standard. These results indicated that the bioactivity of the IL-2 moiety in terms of its ability to support the growth of CTLL-2 cells was maintained in the H520C9sFv-hIL-2 fusion protein.

To determine if the proliferation effects of the H520C9sFv-hIL-2 fusion protein were strictly IL-2-dependent, inhibition studies using IL-2 neutralizing antibodies were performed. For bioactivity neutralization experiments, 2 ng/ml of rhIL-2 and the same concentration of IL-2 in H520C9sFv-hIL-2 were incubated with increasing concentrations of the anti-rhIL-2 neutralizing mAb MAB202 for 2 hr. at 37° C. in a 96-well microtiter plate. As a control, mouse IgG was added to H520C9sFv-hIL-2 instead of MAB202. Following this incubation period, 1×10⁴ CTLL-2 cells were added to each well. The assay mixture, in a total volume of 100 μl/well, was incubated for 24 hr. at 37° C. in a 5% CO₂ incubator and pulsed with 1 μCi/well [³H]-thymidine for the final 12 hr. The cells were then harvested onto glass fiber filters and [³H]-thymidine incorporation was determined.

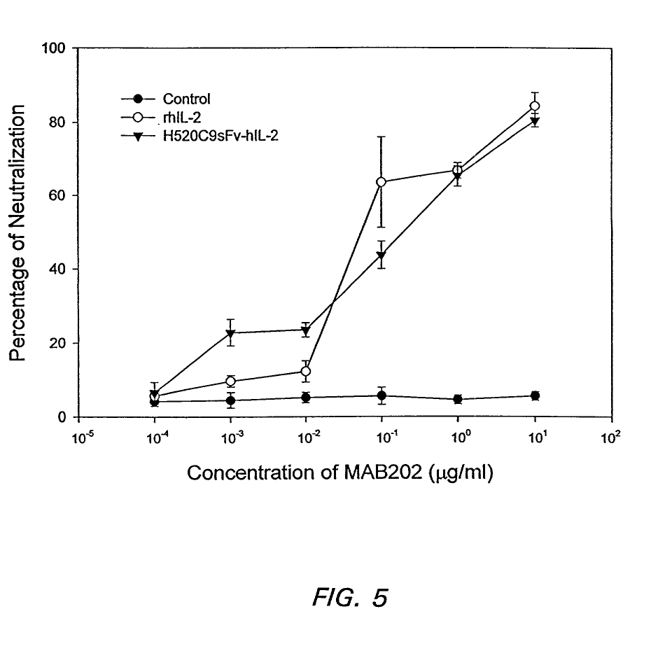

As shown in FIG. 5, the proliferative effects of both rhIL-2 and H520C9sFv-hIL-2 on CTLL-2 cells, as measured by [³H]-thymidine incorporation of the CTLL-2 cells, were inhibited by MAB202. Approximately 50% inhibition of [³H]-thymidine incorporation for 2 ng/ml rhIL-2 or same dose of H520C9sFv-hIL-2 was achieved at a MAB202 concentration of approximately 0.076 and 0.128 μg/ml, respectively. No inhibitory effect was observed when control mouse IgG was added.

In the cytotoxicity assay, peripheral blood mononuclear cells (PBMCs) from healthy individuals were separated on a Histopaque 1077 density gradient (Sigma, St. Louis, Mo.). The PBMCs were cultured in RPMI 1640 medium (Gibco/BRL) supplemented with 10% FCS containing either 1000 IU/ml rhIL-2 or same dose of the H520sFv-hIL-2 fusion protein as determinated by the above CTLL-2 assay for 3 days to produce LAK cells. Cytotoxicity of the resulting LAK cells was assessed by a 4 hr. Calcein AM (Molecular Probe, Eugene, Oreg.) release assay with NK-resistant Daudi cells (ATCC, Rockville, Md.) used as target cells. Each well of a 96-well plate contained 1×10⁴ Daudi cells which had been pre-labelled with fluorescent Calcein AM. To achieve different effector/target cell ratios, an increasing number of LAK cells were added to each well. The total volume of culture medium per well was 0.1 ml. Following coincubation of LAK and Daudi cells at 370 C for 4 hr., the cells were pelleted and the fluorescent intensity value (Valueₛₐₘₚₗₑ) of 50 μl of cell supernatant from each well containing both types of cells was measured with a Cytofluor 2300 fluorescence reader system (Millipore, Bedford, Mass.). The maximal release value (Valueₘₐₓᵢₘₐₗ) of the fluorescent tag was determined by adding an equal volume of 1% (v/v) Triton X-100 to the wells with only target cells. The spontaneous release value (Valueₛₚₒₙₜₐₙₑₒᵤₛ) of the tag was given by the fluorescent intensity of 50 μl of cell supernatant from the wells without any LAK cells. The percent specific lysis for each sample of LAK cells was calculated by the formula (Valueₛₐₘₚₗₑ−Valueₛₚₒₙₜₐₙₑₒᵤₛ)/(Valueₘₐₓᵢₘₐₗ−Valueₛₚₒₙₜₐₙₑₒᵤₛ)×100.

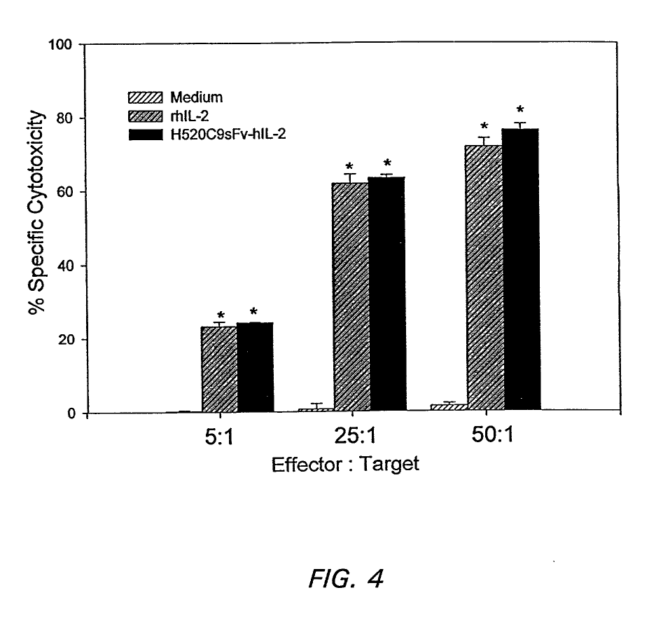

In the cytotoxicity assay directed against target Daudi cells, LAK cells generated by 1000 IU/ml of IL-2 of the H520C9sFv-hIL-2 fusion protein, as determined by the above CTLL-2 assay, demonstrated similar effect in the killing of Daudi cells compared to LAK cells activated by 1000 IU/ml of rhIL-2 (FIG. 4). The specific lysis of Daudi cells by the LAK cells stimulated by the H520C9sFv-hIL-2 and rhIL-2 were 76.3±1.7% and 71.7±2.3%, respectively, at an effector:target ratio of 50: 1. No cytotoxic effect was observed when conditioned medium from control BHK cells transfected with pcDNA3.1(+) was used.

The above examples demonstrate no loss of activity of IL-2 in the H520C9sFv-hIL-2 fusion protein as measured by IL-2-dependent cell proliferation and cytotoxicity assays, compared to rhIL-2 standard. This suggests that the fusion of IL-2 to the C-terminus of the humanized 520C9 sFv does not affect the biological activity of IL-2 and is consistent with previously studies describing the preparation of Fab′-IL-2 and SCA-IL-2 fusion proteins (Fell, et al. J Immunol (1991) 146:2446-2452; and Savage, et al. Br J Cancer (1993) 67:304-310). Without being bound by a particular theory, retention of the IL-2 activity in H520C9sFv-hIL-2 may be partially due to the flexible glycine-rich linker between the C-terminus of sFv and the N-terminus of IL-2 which allows for proper folding of the IL-2 moiety, thereby enabling it to interact with its receptor. This is supported by previous findings with diphtheria toxin-IL-2 fusion proteins demonstrating the importance of the mobility of IL-2 in allowing its interaction to occur effectively (Williams, et al. Prot Engin (1987) 1:493-498; and Kiyokawa, et al. Prot Engin (1991) 4:463-468).

### EXAMPLE 6

### Scatchard Analysis and Antigen Binding Specificity

Scatchard binding assays for the original 520C9 mAb, the humanized 520C9 sFv fragment and the proteolytically cleaved Fab fragment of 520C9 were performed on SK-Br-3 cells as described previously (Ring et al. Cancer Res (1989) 49:3070-3080). Scatchard analysis using SK-Br-3 cells showed that the parental 520C9 mAb had an association constant (Kₐ) of 3.5×10⁸ M⁻¹. Although the Kₐ of the recombinant humanized 520C9 sFv protein (1.1×10⁸ M⁻¹) was slightly lower than that observed for the parental 520C9 mAb, it was higher than the Kₐ observed for the proteolytically cleaved Fab fragment of 520C9 (6.7×10⁷ M⁻¹). These findings indicate that humanization of the 520C9 fragment does not dramatically alter its affinity for p185.

Antigen-binding activity was measured by indirect ELISA using cultured SKOV3ip1 or HeLa cells. Human ovarian carcinoma SKOV3ip1 cells which express high levels of p185 (Yu et al. Cancer Res (1993) 53:891-898), and HeLa cells which express undetectable levels of p185, were maintained in DMEM containing 10% FCS and used for determination of antigen-binding activity of the H520C9sFv-hIL-2 fusion protein. Cells (1×10⁴) were added to individual wells of a 96-well microtiter plate one day before the assay. Serially diluted samples of the H520C9sFv-hIL-2 or a chemically-conjugated control mAb 520C9-rhIL-2 were added to each well of the plate and incubated at 37 C for 2 hr. After three washes with PBST, EP100 (1:250 dilution) was added to the cells and incubated for 2 hr. Following three washes with PBST, HRP-conjugated goat anti-rabbit IgG polyclonal antibodies (Gibco/BRL) (1:1000 dilution) were added to the cells. After 2 hr incubation, the cells were washed three times with PBST, color was developed with the addition of peroxidase substrate (Bio-Rad, Hercules, Calif.) and the OD of the solution was determined at 405 nM in a microplate reader as described.

To demonstrate specificity for p185 binding, SKOV 3ip1 cells (1×10⁴) grown in 96-well plates were treated with either serially diluted intact 520C9 mAb or normal mouse IgG (0.001 to 10 nM) at 37° C. for 2 hr. prior to the addition of 10 nM H520C9sFv-hIL-2 fusion protein. Indirect ELISA was then performed as described above.

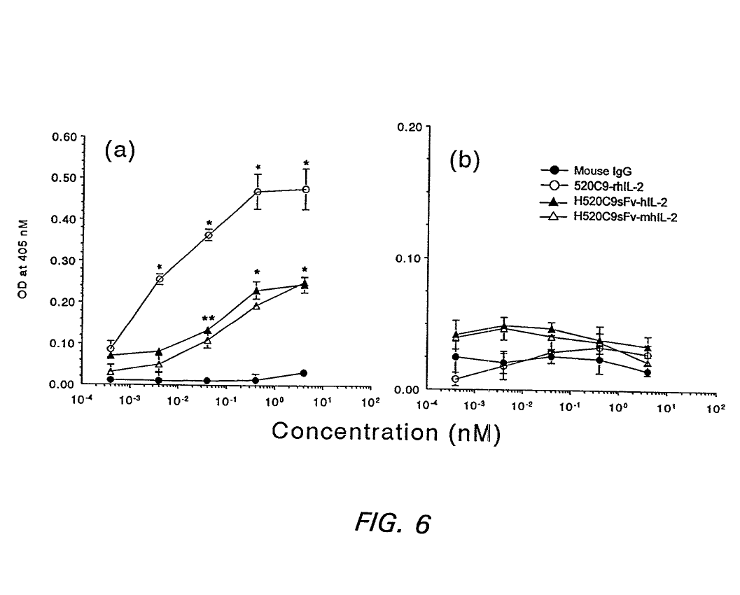

As shown in FIG. 6a, the conditioned medium from BHK cells transfected with either pcDNA-H520C9sFv-hIL-2 or pcDNA-H520C9sFv-mhIL-2 displayed binding activity for p185 on the cell surface of SKOV 3ip1 cells. In contrast, the conditioned media containing the normal or mutant H520C9sFv-hIL-2 fusion protein did not bind to HeLa cells (FIG. 6b), a result consistent with the fact that these cells do not express p185. As a positive control, the parental 520C9 mAb chemically conjugated to rhIL-2 was shown to bind SKOV 3ip1 cells, but not HeLa cells.

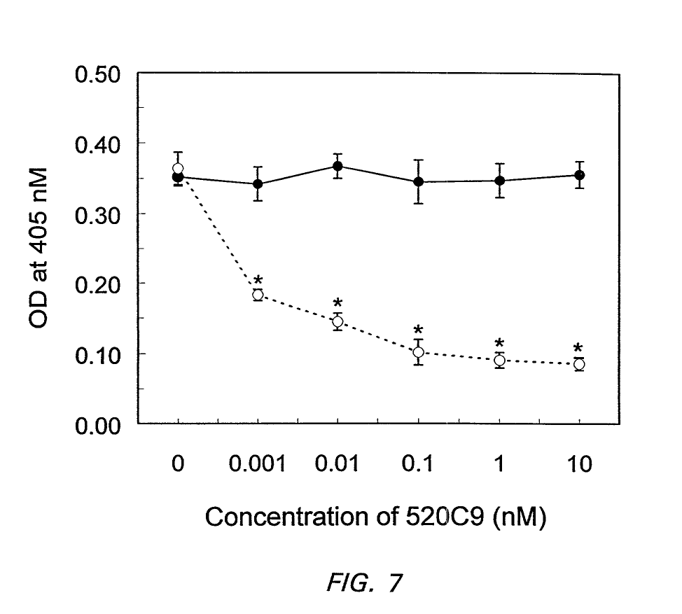

As shown in FIG. 7, 520C9 mAb was able to inhibit the binding of the fusion protein to cells in a dose-dependent manner. In contrast, normal mouse IgG did not inhibit the binding of the fusion protein. Thus, the binding of H520C9sFv-hIL-2 fusion protein was specific for p185.

These findings demonstrate that these fusion proteins specifically bind p185, and suggest that the IL-2 moiety does not impair its antigen-binding properties. As a positive control, the parental 520C9 mAb chemically conjugated to hIL-2 also showed antigen-binding activity. The observation that this chemical immunoconjugate has an apparent higher binding affinity to p185, compared to the recombinant fusion proteins, may result from the monomeric antigen binding valency and one IL-2 molecule in the recombinant fusion proteins compared to the dimeric antigen binding valency and multiple IL-2 molecules in the chemical immunoconjugate.

In antigen-binding ELISAs, EP100 was used to detect the binding activity of H520C9sFv-hIL-2 and H520C9sFv-mhIL-2 to cultured SKOV 3ip1 cells overexpressing p185. Our findings demonstrate that these fusion proteins were able to specifically bind p185, and suggests that the IL-2 moiety does not impair its antigen-binding properties. As a positive control, the parental 520C9 mAb chemically conjugated to hIL-2 also showed antigen-binding activity. The observation that this chemical immunoconjugate has an apparent higher binding affinity to p185, compared to the recombinant fusion proteins, may result from the monomeric antigen binding valency and one IL-2 molecule in the recombinant fusion proteins compared to the dimeric antigen binding valency and multiple IL-2 molecules in the chemical immunoconjugate.

### EXAMPLE 7

### In vivo Efficacy of H520C9sFv-hIL-2

H520C9sFv-hIL-2 was tested in vivo for its ability to retard tumor cell growth as follows. In order to establish an experimental mouse tumor model, B16neu cells, which stably overexpress human HER-2/neu, were grown at 37° C. in DMEM supplemented with 10% fetal calf serum (FCS). Cells grown to 90% confluency were detached with 0.02% EDTA and washed twice with phosphate buffered saline (PBS). Viable cells were determined by trypan blue exclusion staining and the cell concentration was adjusted to 510⁵/mL in PBS. 1×10⁵ B16F0neu cells were then injected subcutaneously into the right inner flank of 6 week-old C57BL mice, previously anaesthetized with Halothane gas. Tumor growth was measured daily in orthogonal directions using calipers. Tumor volume was calculated from the following equation: (minimum measurement)²×(maximum measurement)=mm³.

HEK293 cells, stably overexpressing H520C9sFv-hIL-2 were grown at 37° C. in DMEM supplemented with 10% FCS and 8 μg/mL of G418. Once confluent, the media was removed and a minimal amount of fresh DMEM supplemented with 1% FCS was added. After 3 days, media was collected and the immunoconjugate was purified using an anti-IL-2 affinity column, as described above.

14 days following implantation of B16 cells, mice were placed into two groups. One group of tumor-bearing mice (n=4) received 8 μg of the purified H520C9sFv-hIL-2 protein in 200 μL of Tris-buffered saline (TBS) intravenously daily, for a period of 7 days. The second group of tumor-bearing mice (n=4) received injections of 200 μL of TBS intravenously, for a period of 7 days. Relative changes in tumor volume were measured each day for a period of 10 days following treatment, as described above.

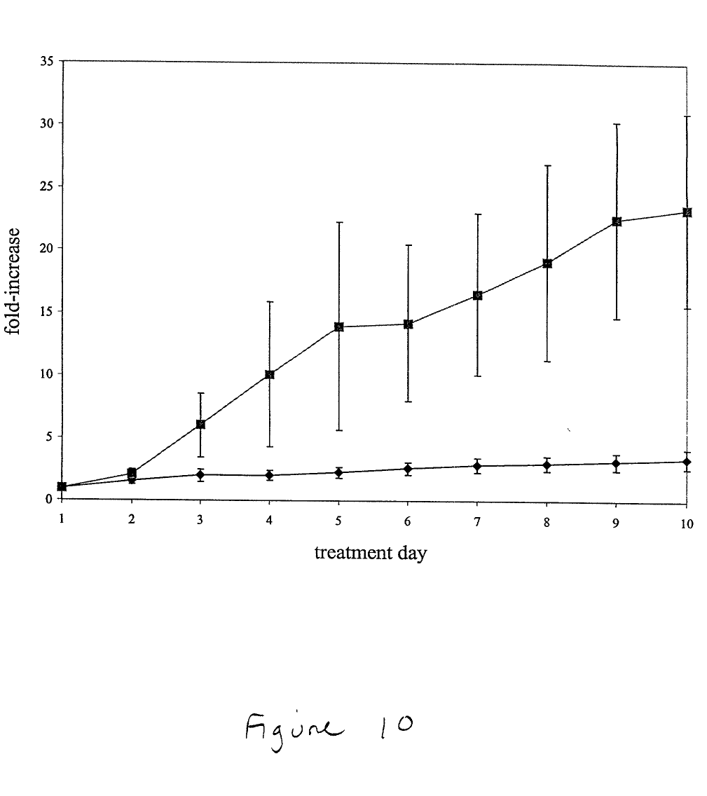

FIG. 10 shows the results of this experiment. As can be seen, the size of the subcutaneous tumor after treatment with H520C9sFv-hIL-2 was relatively unchanged. However, the size of the subcutaneous tumor in mice treated with saline only was dramatically increased 3-5 days post-treatment and continued to grow.

Thus, novel IL-2-anti-p185 immunoconjugates and humanized anti-p185 antibodies, as well as methods of using the same, are disclosed. Although preferred embodiments of the subject invention have been described in some detail, it is understood that obvious variations can be made without departing from the spirit and the scope of the invention as defined by the appended claims.

We claim:

1. A method of inhibiting tumor cell growth in vivo comprising administering to a subject an effective amount of a fusion that comprises an IL-2 polypeptide linked to a p185-specific binding molecule.

2. The method of claim 1, wherein the p185-specific binding molecule portion of the fusion is an antibody.

3. The method of claim 2, wherein the antibody is a monoclonal antibody.

4. The method of claim 2, wherein the antibody is a humanized antibody.

5. The method of claim 2, wherein the antibody is derived from monoclonal antibody 520C9.

6. The method of claim 4, wherein the antibody is derived from monoclonal antibody 520C9.

7. The method of claim 6, wherein the antibody portion of the fusion comprises the amino acid sequence depicted in FIGS. 9A through 9B (SEQ ID NO:______).

8. The method of claim 1, wherein the IL-2 polypeptide is native, human IL-2, as depicted in FIG. 8 (SEQ ID NO:______).

9. The method of claim 1, wherein the IL-2 polypeptide is a biologically active human IL-2 analog.

10. The method of claim 9, wherein the human IL-2 analog comprises an amino acid substitution of cysteine to serine at amino acid 125, numbered relative to FIG. 8 (SEQ ID NO:_____).

11. The method of claim 3, wherein the IL-2 polypeptide is native, human IL-2, as depicted in FIG. 8 (SEQ ID NO:_____).

12. A method of inhibiting tumor cell growth in vivo comprising administering to a subject an effective amount of a fusion protein comprising the amino acid sequence depicted in FIGS. 11A through 11B (SEQ ID NO:_____), or a sequence having at least about 80% sequence identity thereto.

13. The method of claim 12, wherein the fusion protein comprises the amino acid sequence depicted in FIGS. 11A through 11B (SEQ ID NO:______).

14. An immunoconjugate comprising a humanized anti-p185 antibody linked to an IL-2 polypeptide.

15. The immunoconjugate of claim 14, wherein the humanized anti-p185 antibody is derived from monoclonal antibody 520C9.

16. The immunoconjugate of claim 15, wherein the humanized anti-p185 antibody comprises the amino acid sequence depicted in FIGS. 9A through 9B (SEQ ID NO:______).

17. The immunoconjugate of claim 14, wherein the IL-2 polypeptide is native, human IL-2, as depicted in FIG. 8 (SEQ ID NO:______).

18. The immunoconjugate of claim 14, wherein the IL-2 portion of the fusion is a biologically active human IL-2 analog.

19. The immunoconjugate of claim 18, wherein the human IL-2 analog comprises an amino acid substitution of cysteine to serine at amino acid 125, numbered relative to FIG. 8 (SEQ ID NO:_____).

20. The immunoconjugate of claim 14, wherein the humanized anti-p185 antibody and the IL-2 polypeptide are a fusion protein produced by recombinant expression of a chimeric gene encoding the fusion protein.

21. The immunoconjugate of claim 14, wherein the humanized anti-p185 antibody is chemically linked to the IL-2 polypeptide.

22. The immunoconjugate of claim 14, wherein the C-terminus of the humanized anti-p185 antibody is linked to the N-terminus of the IL-2 polypeptide.

23. The immunoconjugate of claim 14, wherein the N-terminus of the humanized anti-p185 antibody is linked to the C-terminus of the IL-2 polypeptide.

24. An immunoconjugate comprising the amino acid sequence depicted in FIGS. 11A through 11B (SEQ ID NO:______), or a sequence having at least about 80% sequence identity thereto.

25. The immunoconjugate of claim 24, comprising the amino acid sequence depicted in FIGS. 11A through 11B (SEQ ID NO:______).

26. A polynucleotide encoding the immunoconjugate of claim 14.

27. A polynucleotide encoding the immunoconjugate of claim 24.

28. A polynucleotide encoding the immunoconjugate of claim 25.

29. A recombinant vector comprising:
(a) the polynucleotide of claim 26; and
(b) control elements that are operably linked to said polynucleotide whereby a coding sequence within said polynucleotide can be transcribed and translated in a host cell, and at least one of said control elements is heterologous to said coding sequence.

30. A recombinant vector comprising:
(a) the polynucleotide of claim 27; and
(b) control elements that are operably linked to said polynucleotide whereby a coding sequence within said polynucleotide can be transcribed and translated in a host cell, and at least one of said control elements is heterologous to said coding sequence.

31. A recombinant vector comprising:
(a) the polynucleotide of claim 28; and
(b) control elements that are operably linked to said polynucleotide whereby a coding sequence within said polynucleotide can be transcribed and translated in a host cell, and at least one of said control elements is heterologous to said coding sequence.

32. A host cell transformed with the recombinant vector of claim 29.

33. A host cell transformed with the recombinant vector of claim 30.

34. A host cell transformed with the recombinant vector of claim 31.

35. A method of producing an immunoconjugate comprising:
(a) providing a population of host cells according to claim 32; and
(b) culturing said population of cells under conditions whereby the immunoconjugate encoded by the coding sequence present in said recombinant vector is expressed.

36. A method of producing an immunoconjugate comprising:
(a) providing a population of host cells according to claim 33; and
(b) culturing said population of cells under conditions whereby the immunoconjugate encoded by the coding sequence present in said recombinant vector is expressed.

37. A method of producing an immunoconjugate comprising:
(a) providing a population of host cells according to claim 34; and
(b) culturing said population of cells under conditions whereby the immunoconjugate encoded by the coding sequence present in said recombinant vector is expressed.

38. A method of producing an immunoconjugte comprising:
(a) providing a humanized anti-p185 antibody; and
(b) chemically conjugating the humanized anti-p185 antibody to an IL-2 polypeptide.

39. A humanized anti-p185 antibody comprising the amino acid sequence depicted in FIGS. 9A through 9B.

40. A polynucleotide encoding the humanized anti-p185 antibody of claim 39.

41. A recombinant vector comprising:
(a) the polynucleotide of claim 40; and
(b) control elements that are operably linked to said polynucleotide whereby a coding sequence within said polynucleotide can be transcribed and translated in a host cell, and at least one of said control elements is heterologous to said coding sequence.

42. A host cell transformed with the recombinant vector of claim 41.

43. A method of producing a humanized anti-p185 antibody comprising:
(a) providing a population of host cells according to claim 42; and
(b) culturing said population of cells under conditions whereby the anti-p185 antibody encoded by the coding sequence present in said recombinant vector is expressed.
